(19) United States
(12) Patent Application Publication
(10) Pub. No.: US 2003/0104493 A1
(43) Pub. Date: Jun. 5, 2003

(54) Method for predicting an increased likelihood of antiphospholipid syndrome in a patient

(76) Inventors: Thomas L. Ortel, Durham, NC (US); Zuowei Su, Durham, NC (US); Paul J. Braun, Durham, NC (US); Liliana Tejidor, Raleigh, NC (US)

Correspondence Address: JUDITH ROESLER, bioMerieux, Inc., 100 Rodolphe Street, DURHAM, NC, 27712, US

(21) Appl. No.: 10/185,186

(22) Filed: Jun. 28, 2002

Related U.S. Application Data
(60) Provisional application No. 60/302,261, filed on Jun. 29, 2001.
(60) Provisional application No. 60/318,755, filed on Sep. 11, 2001.

(51) Int. Cl. G01N 33/53

(52) U.S. Cl. 435/7.9

(57) ABSTRACT

A method for predicting that an individual has antiphospholipid syndrome or an increased likelihood of having antiphospholipid syndrome, includes: a) providing a test sample from an individual; b) combining the test sample with phospholipids; c) directing a light beam at the test sample and monitoring light scattering or transmittance over time so as to provide a time-dependent measurement profile; d) determining if a value or a slope at or over a particular time in the time-dependent measurement profile is beyond a corresponding predetermined value or slope threshold; and if the value or slope in the time-dependent measurement profile is beyond the predetermined threshold, then determining that the individual has antiphospholipid syndrome or an increased risk of antiphospholipid syndrome. The phospholipids can be provided as part of a coagulation reagent, or as part of a reagent where coagulation is not activated. Confirmatory assays for particular antibodies to phospholipid binding proteins can be performed.

This application claims priority from U.S. Provisional Application No. 60/302,261 to Ortel, et al. filed Jun. 29, 2001 and to U.S. Provisional Application No. 60/318,755 to Ortel, et al. filed Sep. 11, 2001, each incorporated herein by reference.

### BACKGROUND OF THE INVENTION

1. Field of the Invention

The present invention is in the field of waveform analysis and the predicting of an abnormality in a patient based on the waveform. The waveform can be provided from a coagulometer (or other analyzer) that monitors changes in light transmittance through a test sample over time so as to provide a time-dependent measurement profile or “waveform”. The present invention is also in the field of detecting antiphospholipid syndrome in a patient, and particularly to obtaining a time-dependent measurement profile from a patient sample, and based on the time-dependent measurement profile, predicting an increased likelihood that the patient has antiphospholipid syndrome (APS), or antiphospholipid antibodies (APLA). This invention is also directed towards monitoring individuals based on the time-dependent measurement profile, and/or assessing thrombotic risk as a result of APS and monitoring therapy in these patients

2. Description of Related Art

Traditionally, the reported results for coagulation tests from the clinical laboratory are provided as clot times. Coagulometers are available that determine clot times by monitoring changes in light transmittance as a function of time. One such coagulometer is disclosed in U.S. Pat. No. 5,646,046 to Fischer et al. issued Jul. 8, 1997, the subject matter of which is incorporated herein by reference. The optical data obtained from these analyzers are used to define specific events that occur prior to, during and following initiation of the clotting reaction. Using this approach, the optical data for a PT (prothrombin time) or APTT (activated partial thromboplastin time) assay can be divided into three segments or ‘phases’: a pre-coagulation segment, a coagulation segment, and a post-coagulation segment (FIG. 2). These segments are characterized by a set of parameters that define: (1) the timing of individual events during the reaction; (2) the rate at which these events occur; and (3) the magnitude of the change.

Transmittance waveforms (TW) have been shown to provide useful information for various clinical situations, such as disclosed in U.S. Pat. No. 6,101,449 to Givens et al. issued Aug. 8, 2000, and U.S. Pat. No. 6,321,164 to Braun et al. issued Nov. 20, 2001, the subject matter of each being incorporated herein by reference. As disclosed therein, waveform parameters can be used to predict the presence of heparin or specific factor deficiencies using a neural network model. The magnitude of the waveform signal has also been used to estimate fibrinogen concentrations in plasma samples. These waveform analysis methods can be used in the present invention for screening patients or predicting an increased likelihood that the patient has antiphospholipid syndrome.

In another example (disclosed in WO 01/96864 to Fischer et al. published Dec. 20, 2001, incorporated herein by reference), a “biphasic” change involving the precoagulation phase of the APTT test has been associated with disseminated intravascular coagulation (DIC). This “biphasic” change is characterized by the appearance of a negative slope 1 in the precoagulation phase of the APTT, and is the result of the formation of a precipitate between C-reactive protein (CRP) and a very low density lipoprotein (VLDL). This complex has been named LC-CRP for Lipoprotein Complexed C-Reactive Protein. This negative slope 1 in the APTT was shown to precede the development of abnormalities in standard laboratory tests for DIC (e.g., elevated D-dimer levels), and waveform changes correlated closely with clinical outcomes. As will be seen below, biphasic waveforms for PT (and for APTT) can be useful for predicting that a patient has APLA, and prompting further testing.

Antiphospholipid antibodies (APLA) are a heterogeneous group of autoantibodies with specificity for complexes consisting of phospholipids and phospholipid-binding proteins, primarily β₂GPI and prothrombin. These antibodies are associated with recurrent arterial and venous thromboembolism, and recurrent spontaneous miscarriage. Diagnostic clinical laboratory tests for APLA are most commonly immunological (anticardiolipin) or functional assays (lupus anticoagulants). Several investigators have reported that pathological anticardiolipin antibodies require the presence of a protein cofactor, β₂GPI, which is present in the fetal bovine serum used in the blocking buffer in the anticardiolipin ELISA. Lupus anticoagulants, on the other hand, recognize prothrombin-phospholipid complexes and inhibit phospholipid-dependent coagulation assays. Other antibodies, including anti-β₂GPI antibodies, also contribute to lupus anticoagulant activity. Several studies have demonstrated that antibodies to β₂GPI and prothrombin are associated with an increased thrombotic risk in patients with APLA.

Based on in-vitro reactivity profiles, APA's are divided into two subclasses: 1) anticardiolipin antibodies (ACA) and 2) lupus anticoagulants (LAC). These reactivity profiles have been known since the early 1950's. The existence of phospholipid-reactive antibodies in human sera was first described in patients with positive serologic tests for syphilis, without evidence of infection. The antibodies responsible for the false-positive syphilis test were ultimately found to recognize cardiolipin within the test reagent. In 1952, the first description of phospholipid-dependent coagulation inhibitors in patients with systemic lupus erythematosus (SLE) was published. A paradoxical association between phospholipid dependent coagulation inhibitors and thrombosis was first described in 1963, and the term lupus anticoagulant was proposed 9 years later based on their prevalence in patients with SLE.

ACA's are detected by immunological methods based on binding of the antibodies to anionic or neutral phospholipids. In recent years it has become clear that the actual antigenic target is not the phospholipid surface but rather proteins that bind to these phospholipids, most notably β₂-glycoprotein I and prothrombin. Immunoassays for the direct measurement of anti-β₂-glycoprotein I and anti-prothrombin antibodies are also available.

LAC's are determined by their interference in phospholipid-dependent clotting assays such as the APTT and the DRVVT. LAC's and ACA's may occur independently or may coexist. LAC and ACA activities may be properties of the same antibody, or the activities may be physically separable.

The term antiphospholipid syndrome (APS) has been used to describe the association between the presence of APA's and clinical features like arterial and venous thrombosis, fetal loss and thrombocytopenia. The range of disease associations is broad. APS may exist in the absence of any underlying disorder (primary APS) or the condition may exist against a background of chronic inflammatory disease related to SLE or other autoimmune diseases, or other pathological conditions. However, as used herein, “antiphospholipid syndrome” or “APS” mean a condition of individuals who simply have antiphospholipid antibodies, whether or not any clinical features are present. “Acute risk” as used herein, means an individual with APS who is at an increased risk for having a clinical event due to the APS, such as a miscarriage, a thrombotic event, an autoimmune disorder, thrombocytopenia, SLE, etc.

### SUMMARY OF THE INVENTION

The laboratory diagnosis of antiphospholipid syndrome presents a paradox to the clinician. In spite of their association with thrombosis, traditional screening assays (APTT and PT) usually show prolonged clotting times. Waveform parameters calculated from optical profiles have been shown to provide additional clinically useful information. The examples presented in this invention show that optical waveform profiles obtained from the PT and APTT are useful in the identification of patients with APS.

The present invention is directed to a method for predicting that an individual has or an increased likelihood of having antiphospholipid syndrome, comprising: a) providing a test sample from an individual; b) combining the test sample with phospholipids; c) directing a light beam at the test sample and monitoring light scattering or transmittance over time so as to provide a time-dependent measurement profile; d) determining if a value or a slope at or over a particular time in the time-dependent measurement profile is beyond a corresponding predetermined value or slope threshold; and if the value or slope in the time-dependent measurement profile is beyond the predetermined threshold, then determining that the patient has antiphospholipid syndrome or an increased likelihood of having of antiphospholipid syndrome.

The present invention is also directed to a method for predicting that an individual is at an increased likelihood for having antiphospholipid syndrome, comprising: a) providing a test sample from an individual; b) combining the test sample with a coagulation reagent comprising phospholipids; c) monitoring the formation of fibrin polymerization over time so as to provide a time-dependent measurement profile; d) defining a set of one or more predictor variables which sufficiently define the data of the time-dependent measurement profile; e) deriving a model that represents the relationship between the antiphospholipid syndrome and the one or more predictor variables; and f) utilizing the model of step e) to predict the increased likelihood of having antiphospholipid syndrome in the individual.

The present invention is further directed to a method for predicting antiphospholipid syndrome in an individual from at least one time-dependent measurement profile, comprising: a) combining a test sample from an individual with phospholipids and directing a light beam at a test sample and monitoring light scattering or transmittance over time so as to provide a time-dependent measurement profile; b) defining a set of one or more predictor variables which sufficiently define the data of the time-dependent measurement profile; c) deriving a model that represents the relationship between the antiphospholipid syndrome and the one or more predictor variables; and d) utilizing the model of step c) to predict the existence of antiphospholipid syndrome in the individual.

The present invention is also directed to a method for predicting an increased risk of thrombosis in a test subject, comprising: a) providing a test sample from an individual; b) combining the test sample with phospholipids; c) directing a light beam through the test sample and monitoring the transmittance of light through the sample over time so as to provide a time-dependent measurement profile; d) determining if a value or slope in the time-dependent measurement profile at a particular time is beyond a corresponding predetermined value or slope threshold; and if the value or slope in the time-dependent measurement profile is beyond the predetermined threshold, then determining an increased risk of thrombosis in the test subject.

The present invention is further directed to a method for monitoring the therapy of an individual having antiphospholipid syndrome, comprising: a) providing a test sample from an individual with antiphospholipid syndrome; b) combining the test sample with phospholipids; c) directing light at the test sample and monitoring light reflectance from or transmittance through the test sample over time so as to provide a time-dependent measurement profile; d) determining a value or slope in the time-dependent measurement profile; e) administering therapy to the patient; f) repeating steps a) to d); and g) comparing the values or slopes to each other in order to determine the efficacy of said therapy.

The invention is still further directed to a method for monitoring the therapy of a patient having antiphospholipid syndrome, comprising: a) providing a test sample from an individual with antiphospholipid syndrome; b) combining the test sample with a coagulation reagent comprising phospholipids c) monitoring the formation of fibrin polymerization over time so as to provide a time-dependent measurement profile; d) determining a value or slope in the time-dependent measurement profile prior to initiation of clot formation; e) administering therapy to the patient based on the value or slope determined.

The present invention is also directed to a method for categorizing an individual as an acute risk patient within a population of APS patients, comprising: a) providing a test sample from an individual; b) combining the test sample with phospholipids; c) directing a light beam at the test sample and monitoring light scattering or transmittance over time so as to provide a time-dependent measurement profile; d) determining if a value or slope at a particular time in the time-dependent measurement profile is beyond a corresponding predetermined value or slope threshold; and if the value or slope in the time-dependent measurement profile is beyond the predetermined threshold, then determining that the APS patient is an acute risk patient.

The present invention is also directed to a method for indirectly measuring a level of antiphospholipid antibodies in a test sample from a test subject with antiphospholipid syndrome, comprising: a) providing a test sample from an individual; b) combining the test sample with phospholipids; c) directing a light beam at the test sample and monitoring light scattering or transmittance over time so as to provide a time-dependent measurement profile; d) determining the value or slope at a particular time in the time-dependent measurement profile; and correlating the value or slope to a level of antiphospholipid antibodies in the test sample.

The invention is yet further directed to a method for predicting that an individual is at an increased likelihood for having antiphospholipid syndrome, comprising: a) providing a test sample from an individual; b) combining the test sample with an APTT reagent; c) monitoring the formation of fibrin polymerization over time so as to provide a time-dependent measurement profile; d) defining a set of one or more predictor variables which sufficiently define the data of the time-dependent measurement profile; e) deriving a model that represents the relationship between the antiphospholipid syndrome and the one or more predictor variables; and f) utilizing the model of step e) to predict the increased likelihood of antiphospholipid syndrome in the individual.

In another embodiment of the invention, a method for determining an increased risk of antiphospholipid syndrome, comprises a) adding an APTT reagent to a patient test sample; b) performing a time dependent measurement profile on the test sample; c) determining whether the profile exhibits a slope or value beyond a predetermined threshhold prior to initiation of clot formation, and if so; d) repeating steps (a) to (c) except with an APTT reagent not comprising calcium so as to confirm the determination of APS (or an increased likelihood of APS) is the profile again exhibits a slope or value beyond a predetermined threshold.

In yet another embodiment of the invention, a method for monitoring an individual, comprises: a) providing a test sample from an individual; b) combining the test sample with phospholipids; c) directing a light beam at the test sample and monitoring light scattering or transmittance over time so as to provide a time-dependent measurement profile; d) determining if a value or a slope at or over a particular time in the time-dependent measurement profile is beyond a corresponding predetermined value or slope threshold; and if the value or slope in the time-dependent measurement profile is beyond the predetermined threshold, then determining that the patient has antiphospholipid syndrome or an increased risk of antiphospholipid syndrome.

### BRIEF DESCRIPTION OF THE DRAWINGS

FIG. 1. This figure shows that the slope_{—}1 change identified 26 out of 41 (63%) patients with APLA.

FIG. 2. This figure shows optical transmittance vs. time for a PT or APTT assay of a normal specimen, including first and second derivatives of transmittance.

FIGS. 3A and 3B. This figure illustrates the distribution of APTT clot times and slope 1 results from patients and controls with and without oral anticoagulant therapy.

FIGS. 4A and 4B. This figure shows the distribution of PT clot times and slope 1 results with Simplastin® L from patients and controls with and without oral anticoagulant therapy.

FIG. 5. This figure illustrates transmittance waveform profiles of PT assays from normal and APLA patient plasma samples.

FIG. 6. This figure shows the effect of heparin on PT and PT slope 1 values. Pooled normal plasma was spiked with pork heparin at concentrations of 0.1, 0.4, 0.6, 0.8, 1.0 and 10 U/ml.

FIG. 7. This figure shows the effect of the addition of a thrombin inhibitor hirudin on PT slope 1 of an APLA patient plasma, demonstrating that the change is independent of thrombin generation.

FIG. 8a illustrates slope 1 for normal and APLA patients when a PT reagent (Simplastin L) is added to patients' plasma, and FIG. 8b shows slope 1 values for normal and APLA patients when a phospholipid mixture is added to patients' plasma samples;

FIG. 9. This figure illustrates the effect of addition of a detergent (Triton X-100) on the PT slope 1 of an APLA patient plasma.

FIGS. 10A to 10C Shows transmittance waveform profiles for PT assays (Simplastin® L) from a) a normal TW; b) a TW with negative slope 1 from a APLA patient's plasma and; c) a TW from the same APLA patient after removal of total IgG.

FIG. 11. This figure shows transmittance waveform profiles of PT and APTT assays from IgG-depleted normal plasmas spiked with total IgG from an APLA patient with; a) normal PT TW before adding IgG; b) abnormal PT TW from the same donor plasma with APLA patient IgG added at 8 mg/mL, showing a negative slope 1; c) normal APTT TW before adding IgG and; d) prolonged APTT TW from the same donor plasma with added APLA patient IgG (8 mg/mL) showing a normal slope 1.

FIGS. 12A to 12D. Figure showing transmittance waveform profiles of PT and APTT assays from IgG-depleted orally anticoagulated plasmas spiked with total IgG from APLA patient with; a) PT TW before adding IgG showing a prolonged PT clot time; b) showing a negative slope 1, an abnormal PT TW from the same donor plasma with APLA patient IgG added; c) APTT TW before adding IgG and; d) prolonged APTT clot time with normal slope 1 from the same donor plasma with APLA patient IgG added.

FIG. 13. Shows the effect of APLA IgG on the international normalized ratios (INRs) INRs in IgG-depleted plasma from 6 controls who were receiving warfarin.

FIG. 14a illustrates the correlation between anti-β₂ glycoprotein antibody and Prothrombin Time slope 1, and FIG. 14b illustrates the correlation between levels of anticardiolipin antibody and Prothrombin Time slope 1.

FIG. 15 is a chart that shows that when total IgG was used in place of plasma in a PT-based assay, only two IgG samples displayed an abnormal precoagulation phase compared to the normal donor samples.

FIG. 16 illustrates that of the plasma proteins listed, only prothrombin and β₂GPI contributed to the generation of abnormal profiles in the IgG waveform assay.

FIGS. 17A and 17B show the IgG waveform assay results for nine APS patients and two normal donors in the presence of increasing concentrations of prothrombin and β₂GPI.

FIG. 18 shows that for one test sample, the non-phospholipid-binding β₂GPI did not induce an abnormal IgG waveform when tested at the same concentrations as its wild type counterpart in the presence of IgG from a particular test sample even though the antibody from this individual bound to the cleaved β₂GPI in an ELISA.

FIGS. 19A and 19B show that in the presence of β₂GPI, varying degrees of discrimination between APLA test samples and normal can be achieved depending upon the PT reagent used.

FIG. 20 illustrates the discriminatory ability of a simple PC:PS (75:25) phospholipid mixture;

FIG. 21 illustrates the improved discriminatory ability of Simplastin L and various PE:PC:PS phospholipid mixtures; and

FIG. 22 illustrates the sensitivity to slope_{—}1 of various thromboplastins.

### DESCRIPTION OF THE PREFERRED EMBODIMENTS

In the present invention, change in light transmittance in a specimen, due to the formation of a complex, is detected as a negative slope (beyond a predetermined threshold) prior to initiation of coagulation in a test sample. This change is indicative of the increased likelihood of antiphospholipid antibodies in the sample being tested. As will be discussed further herein, this initial slope, at times referred to herein as Slope_{—}1, can also be used to distinguish between pathological and non-pathological antiphospholipid antibodies.

When used herein, the term “monitor” or “monitoring” means screening a patient for APS, detecting APLA, diagnosing an individual as having APS, determining the severity of the APS condition of the patient, determining the pathology of the APS condition of the individual, or following the progression or regression of an individual's condition. As used herein, “antiphospholipid syndrome” and “APS” mean a condition where an individual has antiphospholipid antibodies. And the terms “antiphospholipid antibodies” or “APLA” is used herein to mean at least a subset of all antiphospholipid antibodies inclusive of one or more different types of antiphospholipid antibodies. Also, the terms “sample” or “test sample” mean a blood, plasma or serum sample. Also, when the term “beyond” is used herein (as in “determining if a value or a slope at or over a particular time in the time-dependent measurement profile is beyond a corresponding predetermined value or slope threshold”) it is meant to mean that the absolute value of the slope or other value that is measured is greater than the absolute value of the threshold slope of other value. In addition, a “time dependent measurement” is used herein to denote a measurement of a changing parameter in the test sample over time, which changing parameter is determined at multiple points over a period of time so as to result in a “graph” or profile of the changing parameter. The preferred time dependent measurement in the present invention is a measurement of the change in light transmittance through the sample over time. The terms “individual” and “patient” are used interchangeably herein and are not meant to be limited to people under a doctor's care. “Phospholipids” as used herein is a term well known to the skilled in the art. For example, phospholipids that are in the form of vesicles or liposomes can be used for the various methods disclosed herein. A “confirmatory assay” as used herein means an assay that increases the predictive value of the first assay such as one that involves the binding of at least a portion of an antiphospholipid antibody and the detection of such binding.

Materials and Methods

Plasma Samples

Normal donors. Plasma samples from 20 normal donors were obtained to establish the cut-off values for the various APLA antibody levels. Additionally, 6 individuals were recruited and plasma samples were obtained for the IgG spiking assays and for initial IgG purification to establish the normal range in the IgG-mediated light transmittance assay. Two of these donors provided plasma samples for larger scale IgG purification. None of these individuals had a known APLA.

Patients with APLA. Nine patients with APLA and negative PT slope 1 results (using Simplastin L) were recruited. The diagnosis of a APS was made according to the criteria recommended by the Subcommittee on Lupus Anticoagulants/Antiphospholipid Antibodies of the International Society of Thrombosis and Haemostasis. Anticardiolipin antibody IgG levels were determined by ELISA. Samples not used immediately were stored at −70° C. until use.

Reagents

Protein-A Sepharose CL-4B, phosphate buffered saline, pH 7.4 (PBS) packets, ancrod, cardiolipin, annexin V, human serum albumin and other chemicals, were purchased from Sigma Chemical Corporation (St. Louis, Mo.). Human prothrombin, factor IX and factor X, were from Haematologic Technologies (Essex Junction, Vt.). Centricon YM-30 centrifugal filter devices for concentrating IgG preparations were purchased from Millipore Corporation (Bedford, Mass.). Simplastin® L (PT reagent, ISI 2.00) and other reagents used in the coagulation testing were from bioMeriéux, Inc. (Durham, N.C.). Dade Innovin® (PT reagent, ISI 1.00) was from Dade-Behring, Inc. (Newark, Del.).

Determination of Genetic Polymorphisms in β₂GPI, Prothrombin and Factor V

β₂GPI genetic polymorphisms in exon 7 (codon 306) and exon 8 (codon 316) were determined by polymerase chain reaction according to Sanghera, et al. with the following primers: exon 7 forward primer 5′-GTGTAGGTGTACTCATCTACTGT-3′, exon 7 reverse primer 5′-CAAGTGGGAGTCCTAGCTAA-3′, exon 8 forward primer 5′-TTGTTTCTCTTAGAATGTTTAT-3′, exon 8 reverse primer 5′-TGGATGAACAAGAAACAAGTG-3′. Determination of the prothrombin G20210A polymorphism and the Factor V Leiden polymorphism were performed as previously described.

Purification of human plasma β₂GPI

Purification of human plasma β₂GPI was performed according to previously described methods with slight modifications (Izumi, et al., manuscript in review). Briefly, perchloric acid was added to plasma to a final concentration of 1.8% with stirring for 30 min at room temperature. β₂GPI was purified from the supernatant by anion-exchange chromatography and heparin column chromatography. The β₂GPI preparation was checked by sodium dodecyl sulfate-10% polyacrylamide gel electrophoresis (SDS-PAGE) and quantified by ELISA.

A cleaved form of β₂GPI that did not bind to phospholipids was isolated from the partially purified β₂GPI fraction after extended storage at 4° C. The cleavage site was the peptide bond between residues Ala³¹⁴ and Phe³¹⁵, confirmed by protein sequencing. Similar to the plasmin-cleaved β₂GPI (which cleaves between Lys³¹⁷-Thr³¹⁸), this cleaved β₂GPI did not bind to phospholipid.

Immunologic Assays

Anticardiolipin antibody ELI SA. IgG antibodies to cardioli pin were detected by ELISA, as previously described, and anticardiolipin IgG calibrators from Louisville APL Diagnostics, Inc., (Doraville, Ga.), were used to establish a standard curve. An anticardiolipin IgG level of 10 GPL units was established as the cut-off value (one GPL unit is defined as the cardiolipin binding activity of 1 ug/ml of an affinity purified IgG anticardiolipin preparation from a standard serum).

Antiprothrombin and anti-β₂GPI antibody ELISA's. IgG antibodies to human prothrombin were detected as previously described. IgG antibodies to human β₂GPI were detected. The cut-off values for antiprothrombin and anti-β₂GPI were established as the mean obtained from the normal donors plus 3 standard deviations.

Prothrombin Times and Optical Data Parameters

All PT assays were performed in duplicate on an MDA-180® photo-optical coagulometer (bioMerieux, Inc.). In the PT assay, 50 ul of citrated patient plasma was warmed to 37° C. before mixing with 100 ul of the thromboplastin. The reaction was continuously monitored for light transmittance at 580 nm for 150 seconds. Other wavelengths (or multiple wavelengths can be used—and other non-coagulometer analyzers can be used. Except where specifically stated, the thromboplastin used in all experiments was Simplastin L.

During the reaction, a computer algorithm determined clot times and other optical parameters that make up the transmittance waveforms, as described. The slope 1 parameter was defined as the slope of the line beginning at the initiation of the reaction and ending at the onset of coagulation. If the clot time exceeded 25 seconds, or if no clot was detected, the slope 1 parameter was calculated using the optical density at 580 nm at 25 seconds. Transmittance waveforms were downloaded using WET and viewed offline using WIT A.00 software (bioMeriéux, Inc.).

To determine the contribution of fibrinogen to the negative slope 1, defibrinated patient and normal plasmas were prepared as reported. Briefly, 5 ul of 50 U/ml ancrod were added to 1 ml citrated plasma and incubated for 5 min at 37° C. Treated plasmas were centrifuged at 8,000 g to pellet the fibrin clot, and transferred to clean tubes for PT analyses. To determine whether thrombin contributed to the negative slope 1, hirudin (Sigma Chemical Corp., St. Louis, Mo.) was added to APLA patient plasma at final concentrations of 0.1, 0.25, 0.5, 1.0, 10 and 20 U/ml, and hirudin-spiked plasma samples were then used in the PT assays.

Purification of Total IgG

Protein A Sepharose CL-4B columns were prepared according to the manufacturer's instructions. The column was equilibrated with 10 column volumes of PBS. Plasma was thawed and centrifuged for 10 minutes at 10,000 rpm in a Sorvall SS34 rotor, and the supernatant was applied onto the Protein A Sepharose column. IgG-depleted plasma was collected and saved, as described below. The column was then extensively washed with PBS (−50 column volumes or until OD₂₈₀ was zero). The IgG was eluted with 3 column volumes of 0.1 M glycine-HCl (pH 2.5), neutralized in 2 M Tris-HCl (pH 8.0), and the column was then washed with 3 volumes of PBS. The fractions that contained IgG, monitored by OD₂₈₀, were combined and dialyzed against PBS overnight. The dialyzed IgG was concentrated with Centricon YM-30 centrifugal filter devices, and the final IgG concentration was determined with the OD₂₈₀ extinction coefficient for IgG (E_{1%, 1 cm}=14.3). The working concentration of the IgG was adjusted to 40 mg/ml in PBS.

Preparation of IgG-Depleted Plasma and IgG Spiking

IgG-depleted plasma samples from patients and controls were obtained by absorbing the IgG onto a Protein A Sepharose column. To minimize dilution of the plasma with buffer, 8 ml plasma were applied onto a 5 ml Protein A Sepharose column. The first 5-6 ml of plasma flow through was discarded. The following 2-3 ml of plasma were collected as the IgG-depleted plasma. The efficiency of the Protein A column was evaluated by the determination of anticardiolipin IgG antibody levels in the IgG-depleted plasma. After IgG absorption, anticardiolipin IgG antibody levels were undetectable in the IgG-depleted plasma from the two patients with the highest anticardiolipin IgG antibody levels prior to absorption (A003 and A004).

The IgG-depleted control plasmas were spiked with IgG from patients A003 and A004 to a final concentration of 8 mg/ml IgG. Conversely, the IgG-depleted plasma samples from patients A003 and A004 were spiked with IgG from normal donors at the same final concentration. The IgG-spiked plasma samples, together with the complete (unfractionated) plasma and the IgG-depleted plasma samples from patients and controls, were tested in PT assays.

IgG Waveform Assay

The IgG waveform assay substitutes purified IgG at 8 mg/ml (or IgG and plasma protein mixtures) in PBS for the citrated plasma in a PT-based assay on the MDA-180® coagulometer. Fifty microliters of 8 mg/ml IgG (or IgG and plasma protein mix) was warmed to 37° C., mixed with 100 ul of thromboplastin, and then monitored at 580 nm. The slope 1 result was obtained from the first 25 seconds of the waveform profile, since there was no clot formation. An abnormal waveform was defined as greater than 2 standard deviations below the mean obtained with total IgG samples purified from six normal donors.

The following plasma proteins were selected for testing in the IgG waveform assay: prothrombin, β₂GPI, factor IX, factor X and annexin V. Human serum albumin was included as a negative control. Individual plasma proteins were mixed with IgG at their physiological concentrations and at concentrations that were four times the physiological concentrations prior to incubating with thromboplastin (prothrombin, 100 and 400 ug/ml; β₂GPI, 200 and 800 ug/ml; factor IX, 5 and 20 ug/ml; factor X, 10 and 40 ug/ml; annexin V, 4 and 16 ng/ml; and human serum albumin, 40 and 160 mg/ml). For plasma proteins that contributed to the generation of an abnormal waveform, additional concentrations were included to investigate concentration dependence. To test for dependence on phospholipid-binding of the protein, cleaved β₂GPI was used in the same concentrations as native β₂GPI.

Thromboplastin specificity of the IgG waveform assay was determined by comparing results obtained with Simplastin L and Dade Innovin. Purified normal and patient IgG samples were incubated with the individual thromboplastin with or without the presence of β₂GPI or prothrombin.

Statistical Analysis

Primary data were downloaded into an Excel spreadsheet file for analysis (Microsoft Corporation, Redmond, Wash.). Statistical analysis were performed using Prism, v. 3.0 (Graphpad Software, Inc., San Diego, Calif.). Data are expressed as mean±standard deviation (SD). Paired T tests (two-tailed) were used to compare changes in clot time and slope 1 values before and after addition of total IgG from APLA patients to IgG-depleted normal plasmas and IgG-depleted plasmas from orally anticoagulated non-APLA patients, and before and after adding plasma proteins at physiological concentrations to patient total IgG. Statistical significance was defined as p<0.05.

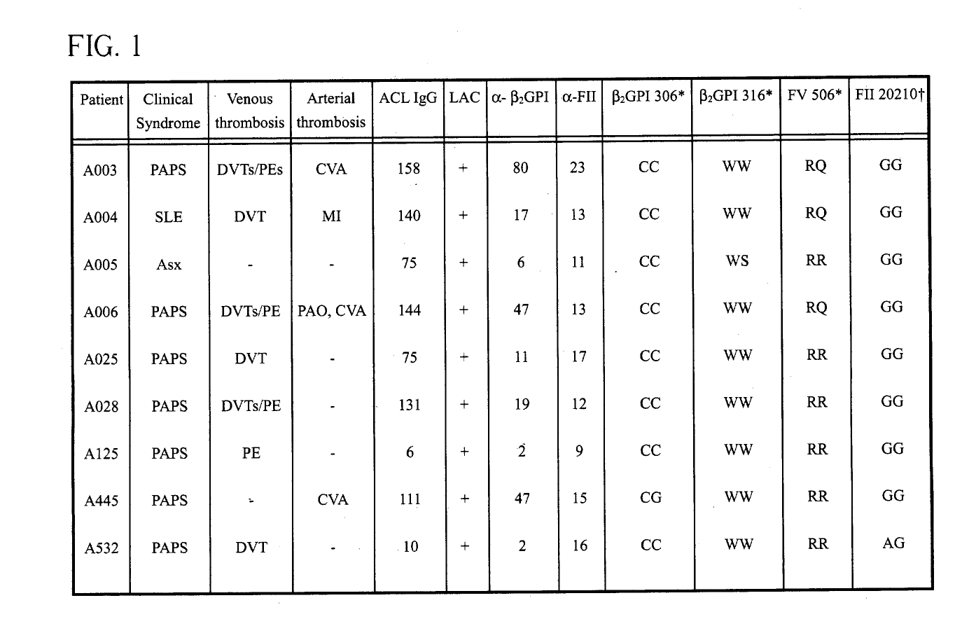

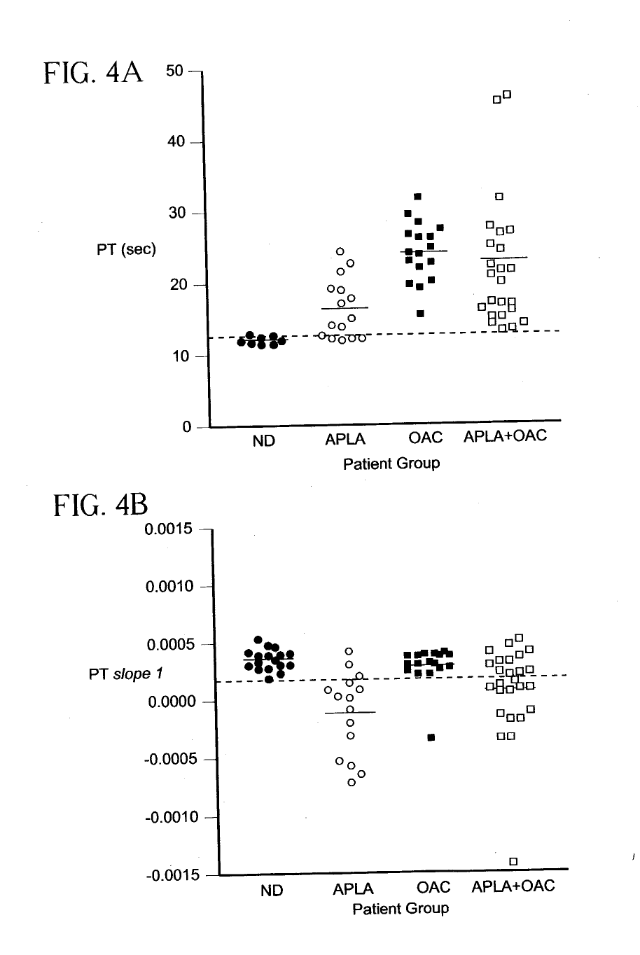

As is shown in FIG. 1 and FIG. 4B, this slope 1 change identified 26 out of 41 (63%) patients with APLA, and this was the only parameter on its own that distinguished the APLA patients from both normal and non-APLA patients on warfarin.

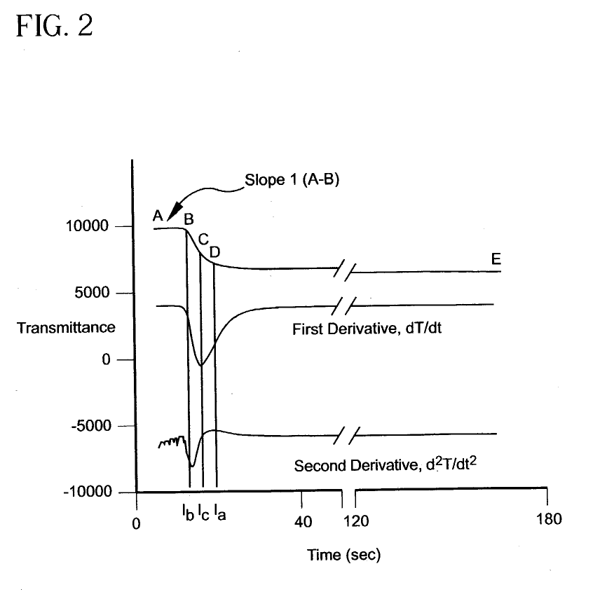

In one embodiment of the invention, a prothrombin time reagent containing phospholipids is mixed with the individual's test sample. This can be accomplished in a number of ways, such as by providing an aliquot of a test sample from a test sample container (e.g. a Vacutainer-type container) that is pierced with an automated probe with the probe aspirating the aliquot of the sample from the sample container. The automated probe is moved to a position over a cuvette and deposited therein. Another automated probe aspirates the reagent from a reagent container and moves to a position over the cuvette in order to deposit the reagent therein. A light beam is transmitted through the cuvette and the transmitted light is detected over time, thus providing a time-dependent measurement profile—in this case a light transmittance profile. If a coagulation reagent (e.g. PT reagent, APTT reagent, TT reagent, DRVVT reagent, tissue factor, snake venom+phospholipids, etc.) is added to the test sample, then a coagulation waveform will result, as can be seen in FIG. 2.

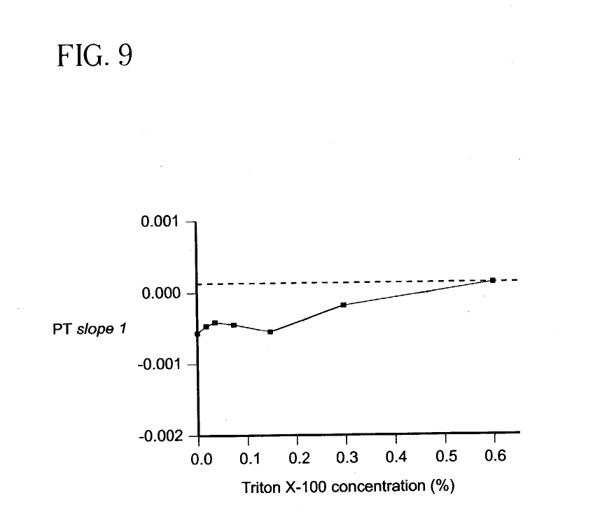

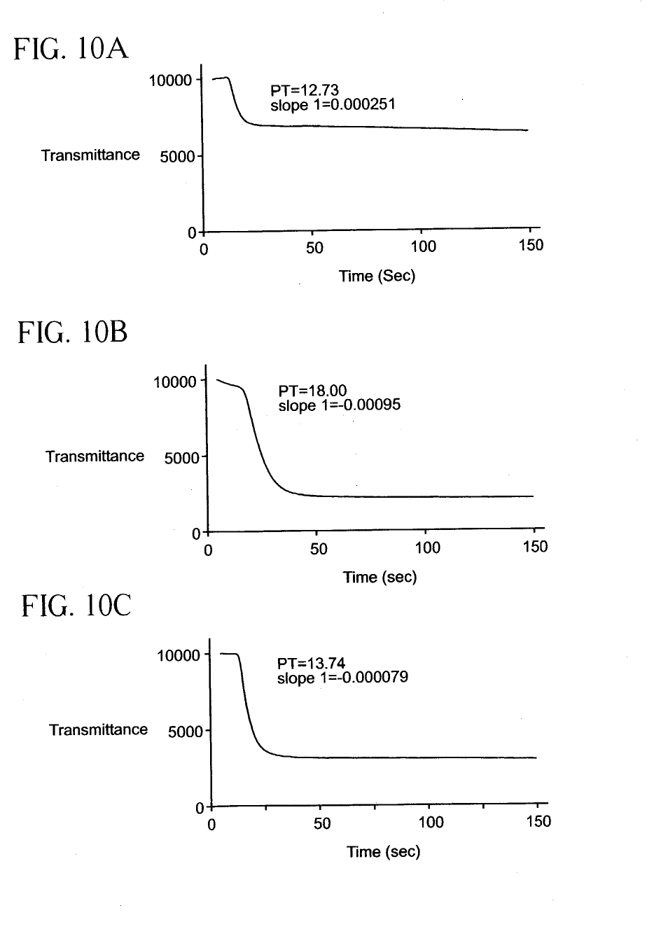

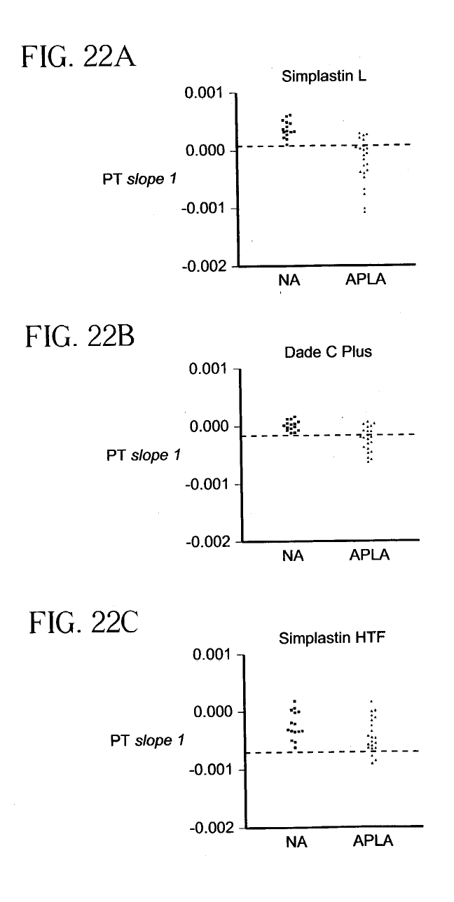

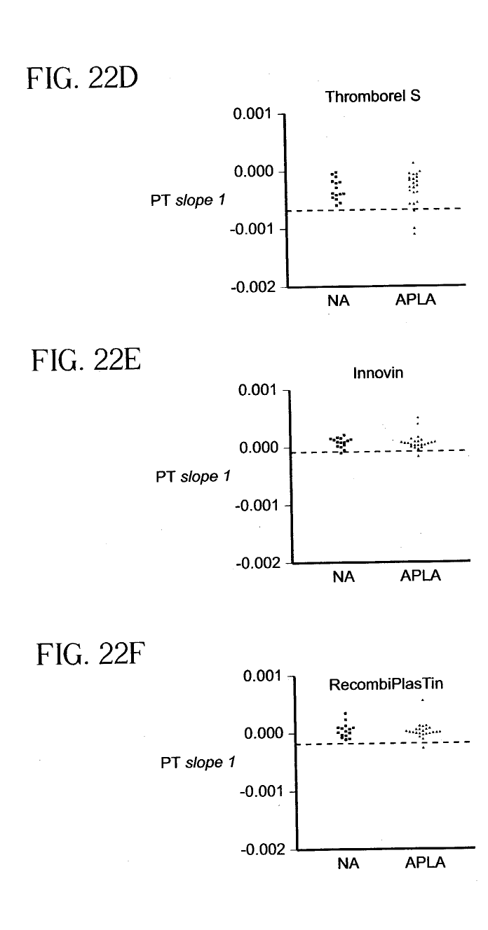

In an embodiment of the invention where a prothrombin time reagent or thromboplastin is used, the reagent can be Simplastin® L, which shows the greatest sensitivity to APLA and best discriminatory ability of the Prothrombin Time reagents evaluated. Other reagents also show sensitivity to APLA (as can be seen in FIG. 22), including HTF (Simplastin R HTF) and Dade C plus. Lipid structures are important for the formation of this complex because as is illustrated in FIG. 9, addition of Triton X-100 abrogated the slope 1 response in a known APLA individual's sample. In another embodiment of the invention, a prothrombin time (PT) reagent is not used, but rather phospholipids with or without a metal cation is combined with the test sample and a slope 1 change as described above is determined.

FIG. 2 shows the optical transmittance vs. time for a Prothrombin Time assay of a normal specimen, including first and second derivatives of transmittance. Events during coagulation are indicated by identifiers A (beginning of signal), B (onset of coagulation), C (midpoint of coagulation), D (end of coagulation) and E (end of signal). The three segments of the reaction in FIG. 2 include the precoagulation segment (A-B), the coagulation segment (B-D), and the post-coagulation segment (D-E). The parameters tB, tC, and tD refer to tmin2, tmin1, and tmax2, respectively, which correspond to coagulation onset, midpoint, and end. Clotting times reported on the MDA® are derived from tmin2. Slope 1 is the slope of the line connecting points A and B (the precoagulation phase), and slope 3 is the slope of the line connecting points D and E (the postcoagulation phase).

Coagulation is not the only event that will cause a decrease in transmittance through the cuvette. Slope 1, that is the initial slope prior to initiation of coagulation (defined as the slope of the line from point A to point B, see arrow in FIG. 2) is a result of an abnormal decrease in light transmittance prior to the onset of coagulation. This initial negative slope is indicative of an increased likelihood of antiphospholipid syndrome, as will be shown in the examples below.

Negative PT Slope1 is Observed in Patients with APLA

Waveform parameters were calculated from PT and APTT optical data from MDA® for normal donor plasmas, patients receiving oral anticoagulants, APLA patients and APLA patients receiving oral anticoagulants. Mean results for PT parameters from these patient groups (Table 1) showed the diagnostic utility of waveform parameters, particularly slope 1 and slope 3 in discriminating APLA populations without being affected by oral anticoagulants that were not also affected by oral anticoagulants. An abnormal slope 1 result (more than SD below the mean of the normal donors) was observed for 63% of the APLA patients (26 of 41), whereas an abnormal slope 3 results was observed for 24% (10 of 41) of APLA patients (FIG. 4 and data not shown).

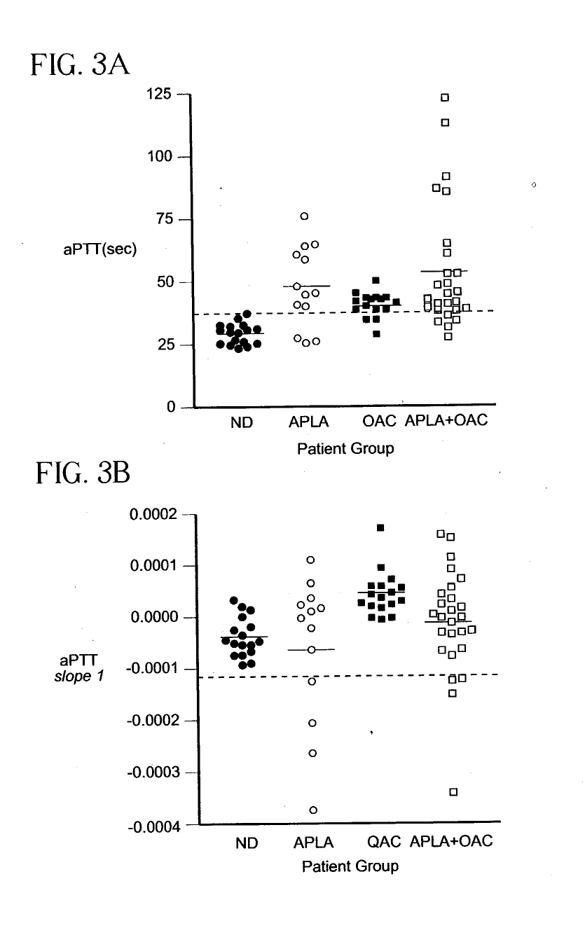

It a coagulation reagent is used in the present invention, a PT reagent is preferred over an APTT reagent, however APTT clot profiles can be used, preferably when more than one clot profile parameter (e.g. clot time, slope_{—}1 and/or slope_{—}3) is used. Mean results for APTT parameters from these patient groups (Table 2) indicated that slope 1 and slope 3 showed diagnostic utility for APLA populations. These parameters were also not affected by oral anticoagulants. Only 15.4% of APLA patients on oral anticoagulants (4 of 26) and 30.8% of APLA patients not on oral anticoagulants (4 of 13) had an abnormally decreased APTT slope 1 value more than 2 SD below the mean for normal donors (FIG. 3). The APTT clot time, which is frequently used as part of testing for APLA, was prolonged in 75.6% of APLA patients (31 of 41), but was also prolonged in 82.4% (14 of 17) of non-APLA patients on oral anticoagulants. These results indicated that PT slope 1 and APTT slope 1 were abnormal in a high in a percentage of APLA patients and these parameters were also useful for APLA patients receiving oral anticoagulants.

More specifically in relation to FIG. 3, this figure illustrates the distribution of APTT clot times and slope 1 results from patients and controls with and without oral anticoagulant therapy. All samples were run with Platelin® L on an MDA® photo-optical coagulometer and transmittance waveform profiles were downloaded for analysis. The APTT clot times were shown in panel A, and the dashed line identified the value that is 2 standard deviations above the mean of the normal donors. The APTT slope 1 results are shown in panel B, and the dashed line identified the value that is 2 standard deviations below the mean. The horizontal solid lines identify the mean value for each subset of individuals. Abbreviations include: ND, normal donors; OAC, oral anticoagulant patients; APLA, antiphospholipid antibody patients not on oral anticoagulant therapy: APLA+OAC, antiphospholipid antibody patients on oral anticoagulant therapy.

As can be seen in FIG. 4, a distribution of PT clot times and slope 1 results with Simplastin® L is shown from patients and controls with and without oral anticoagulant therapy. All samples were run with on an MDA® photo-optical coagulometer and transmittance waveform profiles were downloaded for analysis. The PT clot times as shown in panel A, and the dashed line identifies the value that is 2 standard deviations above the mean of the normal donors. The PT slope 1 results are shown in panel B, and the dashed line identifies the value that is 2 standard deviations below the mean. The horizontal solid lines identify the mean value for each subset of individuals. Abbreviations are the same as for FIG. 3.

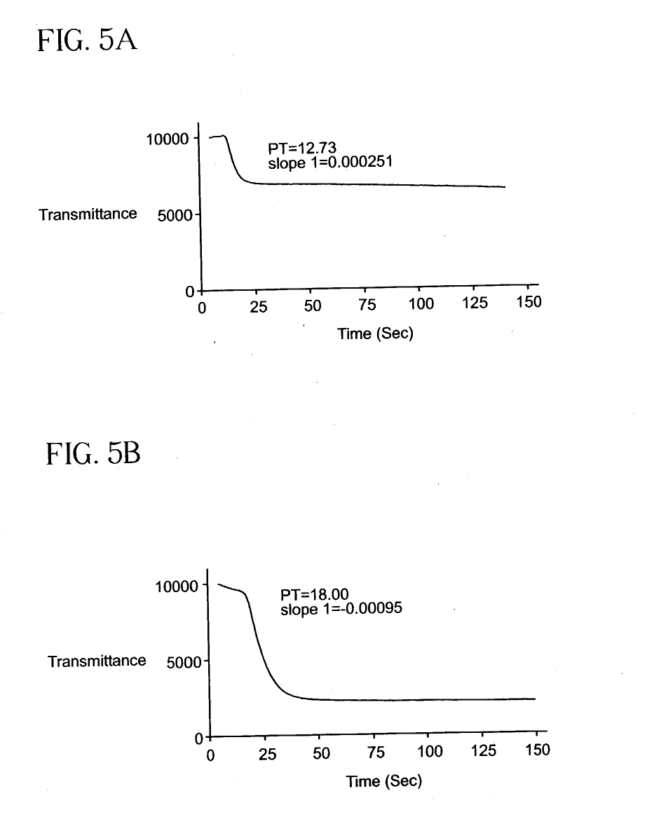

As can next be seen in FIG. 5, transmittance waveform profiles of PT assays are shown from normal and APLA patient plasma samples. Prothrombin times were run with Simplastin® L on the MDA® coagulometer with plasma samples from (A) a normal donor, and (B) an APLA patient not on warfarin therapy.

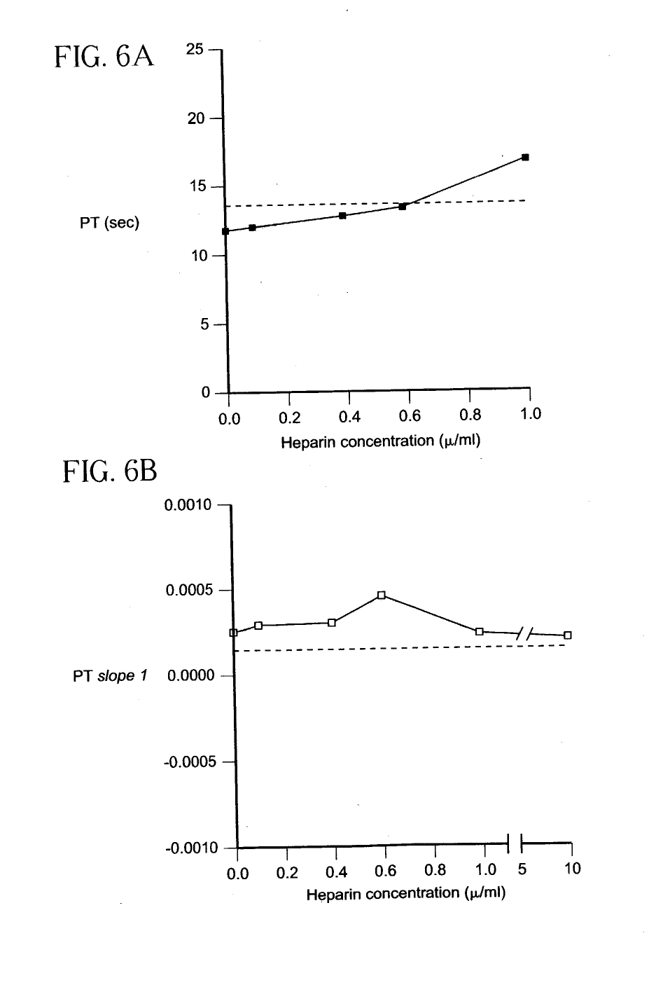

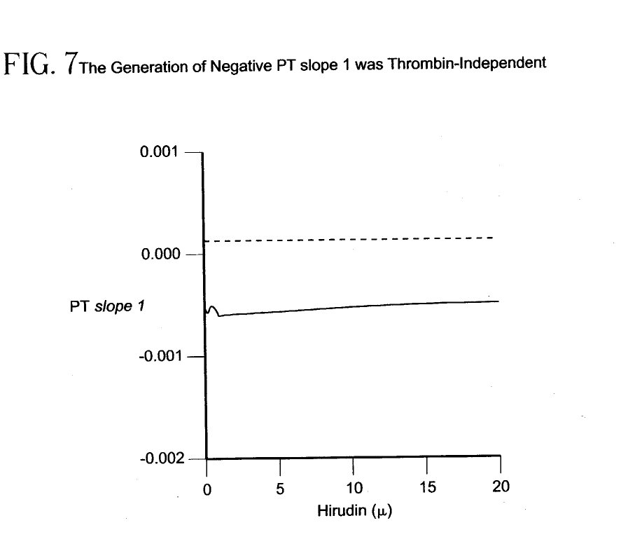

FIG. 6 illustrates the effect of heparin on PT and PT slope 1 values. Pooled normal plasma was spiked with pork heparin at concentrations of 0.1, 0.4, 0.6, 0.8, 1.0 and 10 U/ml. The spiked plasmas were run PT with Simplastin® L on the same MDA® photo-optical coagulometer and transmittance waveform profiles were downloaded for analysis (heparin at 10 U/ml resulted in no clot). In panel A, the dashed line identifies the value that is 2 standard deviations above the mean; on panel B, the dashed line identifies the value that is 2 standard deviations below the mean. FIG. 7 shows the effect of the addition of a thrombin inhibitor hirudin on PT slope 1 of an APLA patient plasma, demonstrating that the change is independent of thrombin generation.

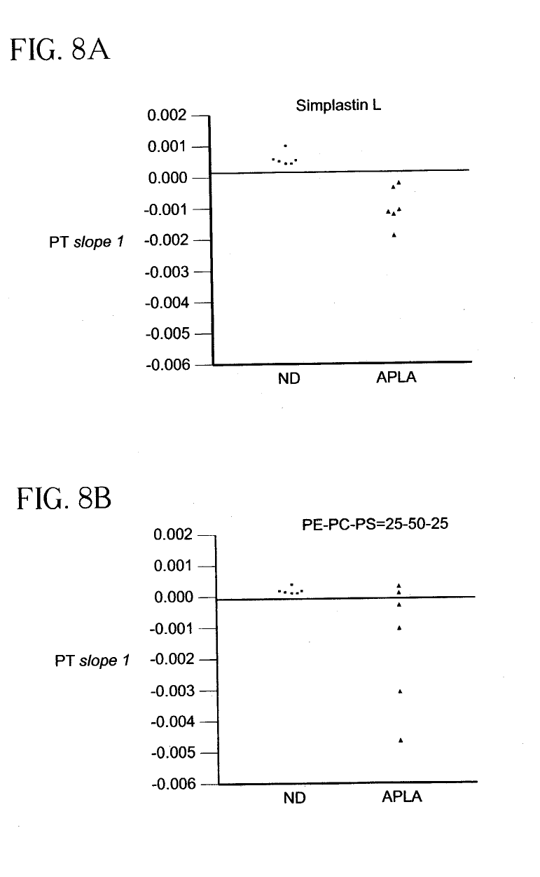

As can be seen in FIG. 8a, slope 1 for normal and APLA patients is shown when a PT reagent (Simplastin L) is added to patients' plasma, and FIG. 8b shows slope 1 values for normal and APLA patients when a phospholipid mixture is added to patients' plasma samples. FIG. 9 illustrates the effect of addition of a detergent (Triton X-100) on the PT slope 1 of an APLA patient plasma. These data show the requirement for phospholipid surfaces. Triton X-100 diminished the slope_{—}1 change in a dose-dependent manner.

TABLE 1
PT clot times and optical parameters with Simplastin ® L.

| | | | Non-APLA | |
|---|---|---|---|---|
| | Normal | APLA | patients on | APLA patients |
| | donors | patients | warfarin | on warfarin |
| Parameter | n = 17 | n = 15 | n = 17 | n = 26 |
| PT clot time | 12.15 ± 0.29 | 16.39 ± 4.26^{†,‡} | 24.31 ± 4.13^{†,‡} | 23.11 ± 10.55^{†} |
| Slope 1 | 0.315 ± 0.09 | −0.174 ± 0.37^{†,‡} | 0.238 ± 0.18 | 0.022 ± 0.39^{†,‡} |
| (×10⁻³) | | | | |
| tmin2 | 12.14 ± 0.33 | 16.41 ± 4.21^{†,‡} | 24.52 ± 4.17^{†} | 23.29 ± 10.68^{†} |
| min2 (×10⁻²) | −0.126 ± 0.02 | −0.130 ± 0.07 | −0.085 ± 0.03^{†} | −0.096 ± 0.04^{†} |
| tmin1 | 13.52 ± 0.32 | 18.23 ± 4.96^{†,‡} | 26.38 ± 4.41^{†} | 25.21 ± 11.11^{†} |
| min1(×10⁻¹) | −0.130 ± 0.03 | −0.148 ± 0.07 | −0.103 ± 0.02^{†} | −0.120 ± 0.04 |
| tmax2 | 14.89 ± 0.38 | 20.19 ± 6.37^{†,‡} | 28.04 ± 4.49^{†} | 26.83 ± 11.24^{†} |
| max2 (×10⁻³) | 0.548 ± 0.09 | 0.512 ± 0.31 | 0.287 ± 0.09^{†} | 0.363 ± 0.18^{†} |
| Slope 3 | −0.109 ± 0.02 | −0.081 ± 0.05^{†} | −0.102 ± 0.04 | −0.103 ± 0.08^{†} |
| (×10⁻³) | | | | |
| delta | 0.312 ± 0.07 | 0.510 ± 0.17^{†} | 0.470 ± 0.10^{†} | 0.510 ± 0.16^{†} |

^{†}Results for the patient groups that significantly differ from the results obtained for the normal donor population.
^{‡}Results for the APLA patients (taking warfarin or not) that significantly differ from the results obtained for the non-APLA patients on warfarin therapy.

TABLE 2
APTT clot times and optical parameters

| | | | Non-APLA | |
|---|---|---|---|---|
| | Normal | APLA | patients on | APLA patients |
| | donors | patients | warfarin | on warfarin |
| Parameter | n = 17 | n = 13* | n = 17 | n = 26 |
| APPT clot time | 28.82 ± 4.04 | 47.73 ± 16.32^{†} | 39.87 ± 4.64^{†} | 53.32 ± 25.38^{†} |
| Slope 1 | −0.039 ± 0.04 | −0.062 ± 0.14^{‡} | 0.043 ± 0.04^{†} | −0.013 ± 0.20^{‡} |
| (×10⁻³) | | | | |
| tmin2 | 28.34 ± 4.11 | 47.32 ± 16.45^{†} | 39.93 ± 4.71^{†} | 53.31 ± 25.55^{†} |
| min2 (×10⁻³) | −0.201 ± 0.03 | −0.183 ± 0.10 | −0.162 ± 0.04^{†} | −0.165 ± 0.06^{†} |
| tmin1 | 31.93 ± 5.01 | 51.83 ± 17.66^{†} | 42.36 ± 4.69^{†} | 57.42 ± 26.86^{†} |
| min1(×10⁻¹) | −0.102 ± 0.02 | −0.096 ± 0.06 | −0.087 ± 0.02^{†} | −0.087 ± 0.04 |
| tmax2 | 37.74 ± 4.74 | 60.62 ± 20.05^{†} | 49.51 ± 4.99^{†} | 65.46 ± 28.30^{†} |
| max2 (×10⁻³) | 0.166 ± 0.03 | 0.130 ± 0.01^{†} | 0.093 ± 0.03^{†} | 0.102 ± 0.06^{†} |
| Slope 3 | −0.016 ± 0.01 | −0.052 ± 0.10^{‡} | −0.059 ± 0.03^{†} | −0.038 ± 0.04^{‡} |
| (×10⁻³) | | | | |
| delta | 0.479 ± 0.09 | 0.693 ± 0.17^{†} | 0.642 ± 0.111^{†} | 0.692 ± 0.15^{†} |

* Two of the APLA patients who were not on warfarin had APTT results that were >150 sec and are not included in these analyses.
^{†}Results for the patient groups that significantly differ from the results obtained for the normal donor population.
^{‡}Results for the APLA patients (taking warfarin or not) that significantly differ from the results obtained for the non-APLA patients on warfarin therapy.

The negative PT Slope 1 in Plasmas from APLA Patients is Due to IgG.

To determine whether patient IgG contributed to the observed abnormalities in the PT slope 1, the PT optical profiles from a normal plasma sample (FIG. 10A) were compared with the PT profiles from APLA patient plasma before (FIG. 10B) and after (FIG. 10C) removal of total IgG. IgG antibodies were removed from plasma for two patients with elevated APLA using Protein A Sepharose CL-4B column chromatography. Removal of total IgG from the APLA patient resulted in almost complete normalization of PT slope 1 (12 fold reduction in absolute value) as well as a greatly shortened clot time, compared to the same plasma before IgG depletion (FIG. 10B and C). This patient was not on oral anticoagulant therapy at the time of testing and did not have an acquired hypoprothrombinemia.

Addition of IgG from APLA Patients to IgG-Depleted Normal Plasma.

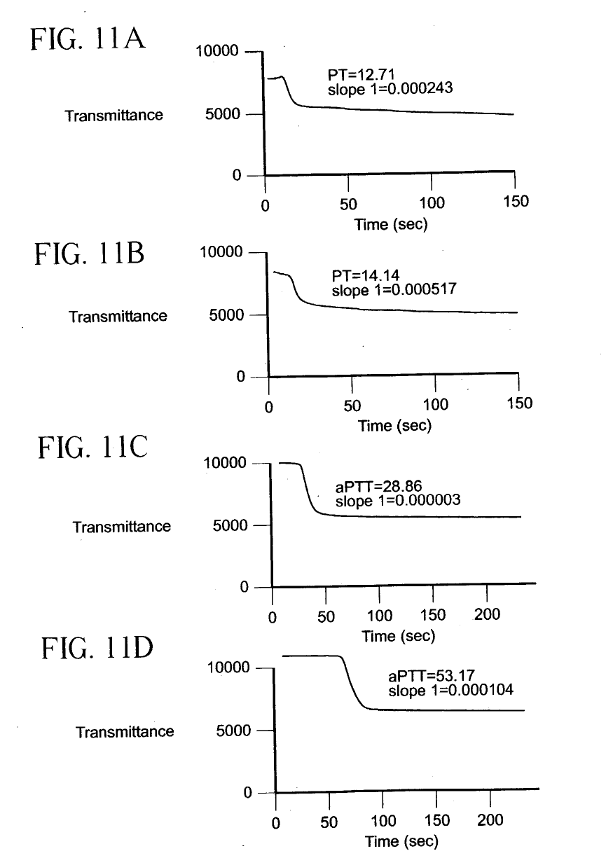

Total IgG purified from two APLA patients was added to IgG-depleted plasma samples from 6 normal donors to give a final concentration of 8 mg/ml. The mean PT slope 1 result for the 6 normal plasmas at baseline was 0.306×10⁻³±0.109×10⁻³, and the mean PT clot time was 12.88±0.23 seconds. After addition of APLA IgG, the mean PT slope 1 was −0.521×10⁻³±0.063×10⁻³ (p<0.0001) and the mean PT clot time was 13.71±0.24 seconds (p<0.0001). The optical profiles for an individual experiment with one normal plasma are shown in FIGS. 11A and 11B for PT and 11C and 11D for APTT assays. These same IgG preparations also caused prolonged APTT clot times, but did not affect the APTT slope 1 results for the 6 IgG-depleted normal plasmas (panels C and D). In contrast, addition of total IgG from normal plasma to IgG-depleted APLA patient plasma did not change the clot times or the slope 1 results (data not shown).

Addition of IgG from APLA patients to IgG-Depleted Plasma from Patients on Warfarin.

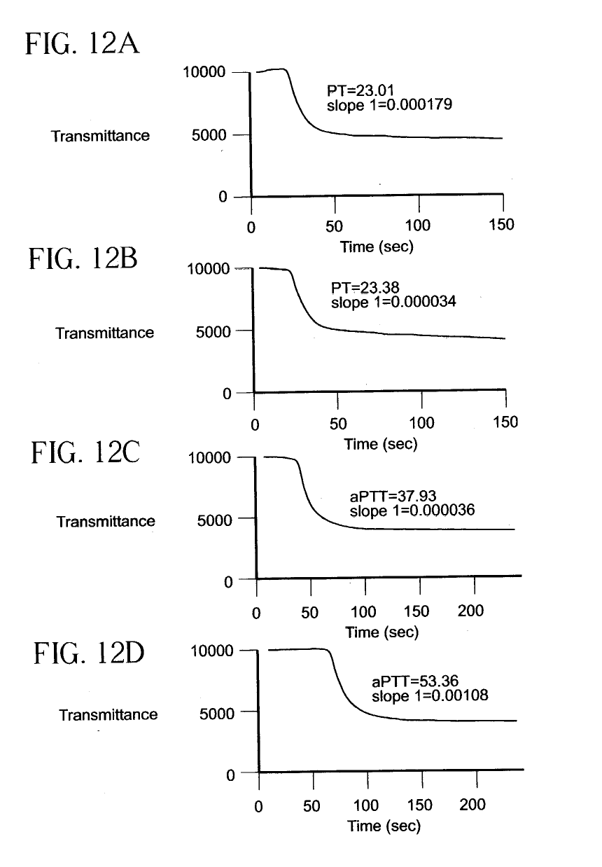

Total IgG from APLA patients was added to IgG-depleted plasma samples from 6 non-APLA patients taking warfarin to give a final concentration of 8 mg/ml IgG. The mean PT slope 1 of the plasma samples from the non-APLA patients on warfarin therapy was 0.231×10⁻³±0.07×10⁻³ and the mean PT clot time was 20.63±2.68 seconds. The mean PT slope 1 of the IgG-depleted plasma samples prior to the addition of APLA IgG was 0.251×10⁻³±0.105×10⁻³, and the mean PT clot time was 20.59±2.829 seconds (p=not.significant). After the addition of APLA IgG, however, the mean PT slope 1 was −0.232×10⁻³±0.0724×10⁻³ (p<0.0001, compared to IgG-depleted plasma), and the mean PT clot time was 22.04±2.829 seconds (p<0.0001). The optical profiles for an individual experiment with one patient on oral anticoagulant therapy are shown in FIGS. 12A and 12B for PT and 12C and 12D for APTT assays. Addition of total IgG purified from a normal donor to IgG-depleted APLA patient plasma did not produce a change in the PT clot time or PT slope 1 results (data not shown).

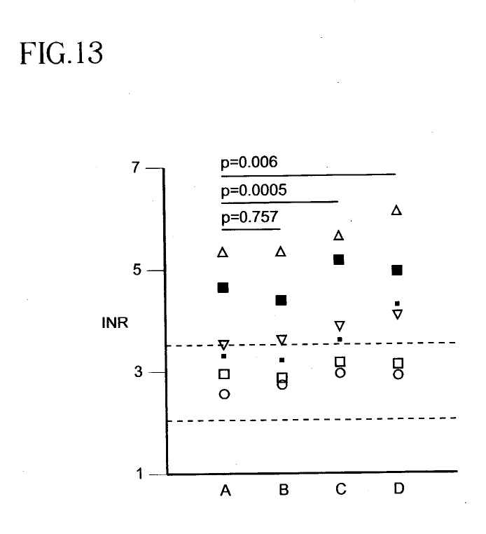

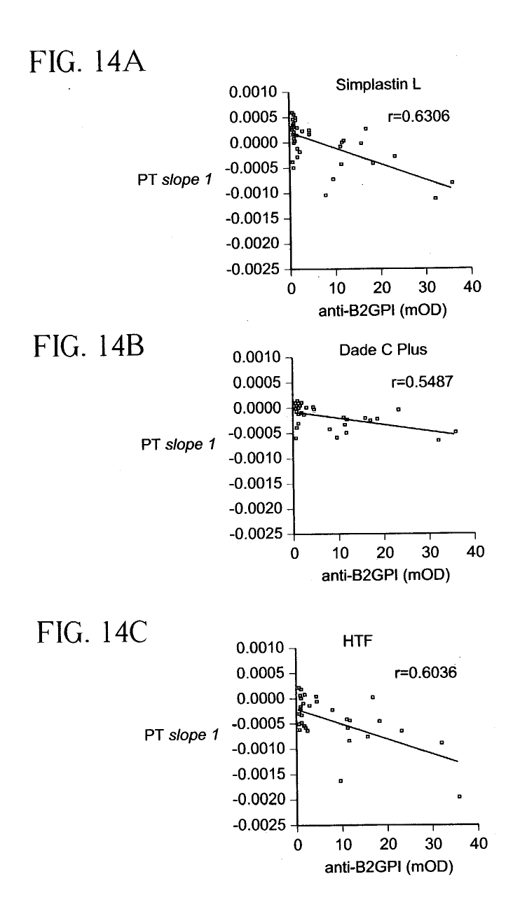

The slight shift in the PT results following addition of total IgG from patients with APLA to IgG-depleted plasma samples from non-APLA patients on warfarin resulted in a statistically significant increase in the INR (FIG. 13). The observed shifts in the INR results were slightly different for the two IgG preparations, consistent with the heterogeneous nature of these antibodies (FIG. 13). For two of the IgG-depleted plasma samples, addition of total IgG from the APLA patients resulted in a shift from a therapeutic INR to a supratherapeutic result (FIG. 13). This result is consistent with the known ability of antiphospholipid antibodies to prolong PT results during oral anticoagulant therapy in some patients. This often results in INR results that do not reflect the actual level of anticoagulation.

Levels of C-Reactive Protein were not Associated with the Presence of Negative PT Slope 1.

The finding that the negative APTT slope 1 in plasma from patients with DIC was due to complex formation between VLDL and CRP, as described in Fischer et al., prompted us to look at levels of CRP in the patients with APLA. Plasma samples from 38 patients with abnormal PT slope 1 values were tested. There was no correlation between the CRP level and the magnitude of the abnormal PT slope 1 result (r=0.1712; p=0.2908). Furthermore, twenty-two of these patients (58%) had a normal CRP level. Therefore, the presence of negative PT slope 1 value in APLA plasma was not related to elevated CRP levels.

The Negative PT Slope 1 Does Not Require Fibrinogen or Thrombin Activity

The negative PT slope 1 is a precoagulation event that occurs before the onset of clot formation. To test whether fibrinogen was required for the generation of a negative PT slope 1, defibrinated plasmas from patients with APLA and normal donors were obtained. Defibrinated plasma samples from normal donors did not clot and did not have a negative PT slope 1 (FIG. 3A). In contrast, although defibrinated patient plasma samples also did not clot, these samples still had a negative PT slope 1 (FIG. 3B). To rule out dependence of the negative PT slope 1 on thrombin activity, APLA patient plasma samples (A003 and A004) were spiked with increasing concentrations of hirudin. Although PT clot times gradually prolonged, the negative PT slope 1 remained unchanged (data not shown).

Purified Patient IgG Did Not Cause an Abnormal IgG Waveform Assay in Most Patients

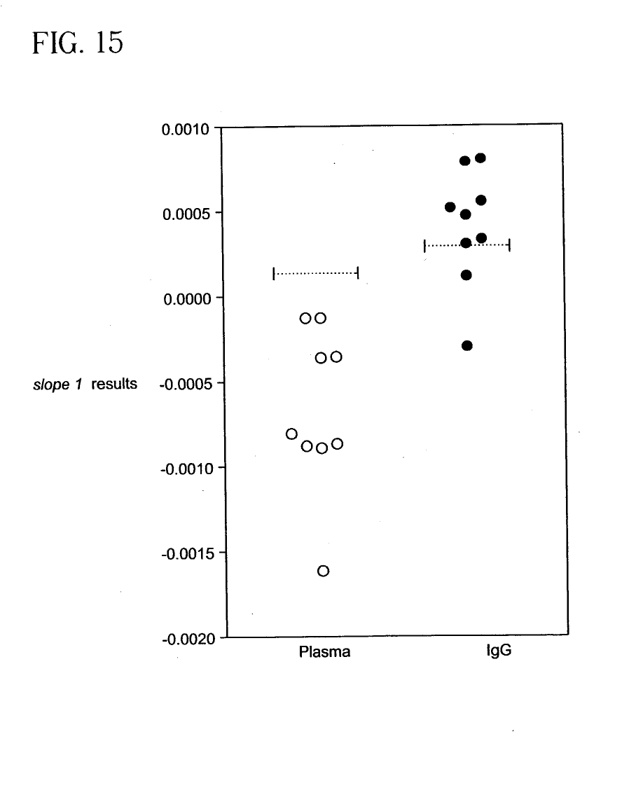

Since the generation of a negative PT slope 1 required neither thrombin nor fibrinogen, we tested whether IgG alone could induce an abnormal precoagulation phase. As shown in FIG. 15, when total IgG was used in place of plasma in the PT-based assays, only two IgG samples (A025 and A445) displayed an abnormal precoagulation phase compared to the normal donor samples, suggesting that additional plasma components were contributing to the abnormal waveform profile.

The Role of Phospholipid-Binding Plasma Proteins in the Generation of an Abnormal IgG Waveform Assay

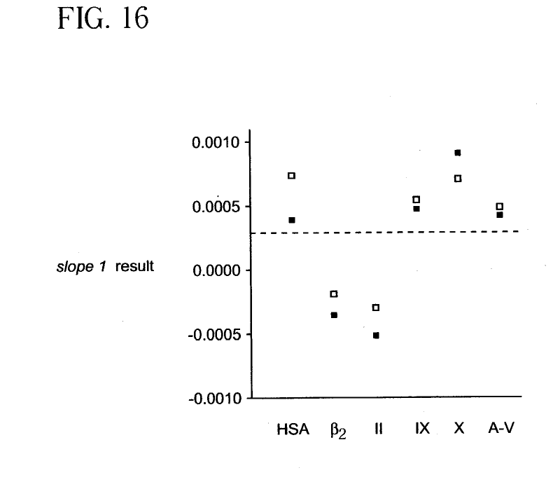

We tested five phospholipid-binding proteins for their contributions to the abnormal precoagulation phase reaction in the IgG waveform assay using purified IgG from patients A003 and A004. Of the plasma proteins tested, only prothrombin and β₂GPI contributed to the generation of abnormal profiles in the IgG waveform assay (FIG. 16). Both proteins caused abnormal IgG waveform results at their physiological concentrations, and slightly more abnormal results at four folds of their physiological concentrations (FIG. 5). Factor IX, factor X and annexin V did not cause an abnormal IgG waveform assay, nor did human serum albumin.

IgG Preparations Differ in their Reactivity with Prothrombin and β₂GPI

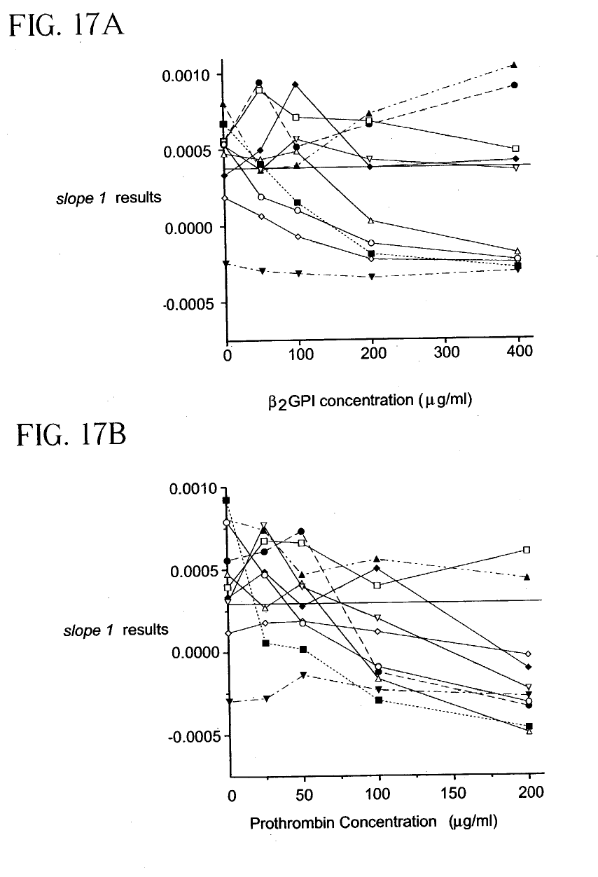

To further define the dependence of the negative PT slope 1 on prothrombin and β₂GPI, we characterized the IgG waveform assay results for the nine patients and two normal donors in the presence of increasing concentrations of prothrombin and β₂GPI. For the two IgG preparations with abnormal IgG waveforms in the absence of phospholipid-binding proteins (A025 and A445), there was an enhancement of the abnormal IgG waveform result for patient A445 with increasing concentrations of β₂GPI (FIG. 17A). There were no effects with added prothrombin for either patient, however (FIG. 17B). Three IgG samples (patients A003, A004 and A028) developed abnormal IgG waveforms in the presence of either β₂GPI (FIG. 6A) or prothrombin (FIG. 6B) in a dose-dependent fashion. All three patients had elevated IgG antibody levels to β₂GPI and prothrombin (FIG. 1). Another three patient IgG samples (patients A005, A006 and A532) showed dependence on prothrombin but not β₂GPI (FIG. 6). All three patients had elevated IgG antiprothrombin levels, but only A006 also had an elevated anti-β₂GPI IgG level (FIG. 1). Lastly, one patient IgG (patient A125) did not induce an abnormal IgG waveform with either prothrombin or β₂GPI. This patient had APS but did not have elevated antibody levels to prothrombin or β₂GPI (FIG. 1). IgG samples from two normal donors did not induce abnormal IgG waveforms either in the presence of or absence of the phospholipid-binding proteins (one normal donor shown in FIG. 6). Prothrombin and β₂GPI alone did not induce an abnormal IgG waveform assay (data not shown).

The abnormal IgG Waveform Required the Binding of β₂GPI to Phospholipids

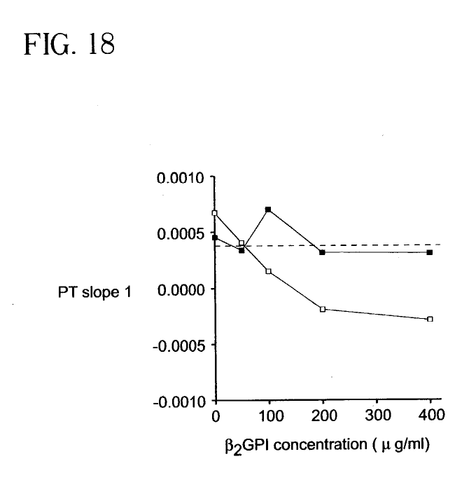

We next investigated whether the protein cofactor had to be able to bind to a phospholipid membrane surface by substituting cleaved β₂GPI for native β₂GPI in the IgG waveform assay. The non-phospholipid-binding β₂GPI did not induce an abnormal IgG waveform when tested at the same concentrations as its wild type counterpart in the presence of A003 IgG (FIG. 18) even though the antibody from this patient bound to the cleaved β₂GPI in an ELISA.

The Abnormal IgG Waveform was Reagent Specific

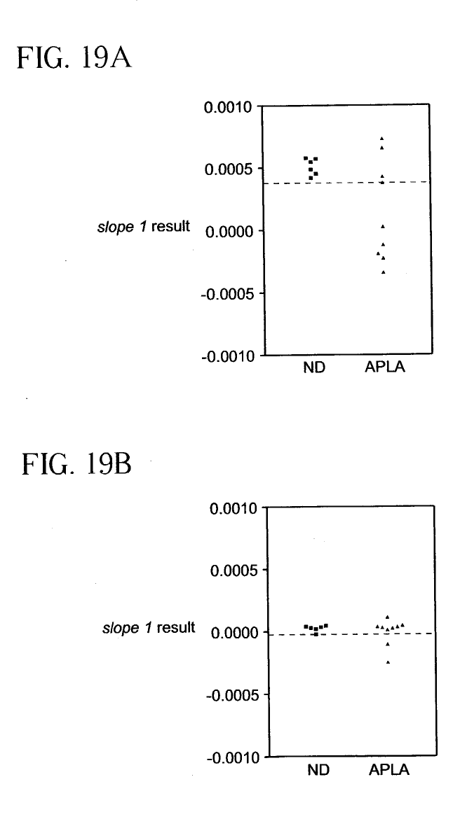

In the presence of 200 ug/ml β₂GPI, an abnormal IgG waveform assay was observed in five of nine patient IgG samples with Simplastin L (Figure 19A). In contrast, only two of nine patient IgG samples had an abnormal IgG waveform assay with the thromboplastin Innovin (FIG. 19B). Of note, these two patients were A025 and A445, both of whom demonstrated a cofactor-independent IgG waveform assay with Simplastin L (FIG. 4). These two patient IgG samples also had abnormal IgG waveform assays with Innovin in the absence of β₂GPI (data not shown).

Correlations with Clinical Outcomes

Four patients had recurrent venous and/or arterial thromboembolic events (A003, A004, A006, and A028). Three of these four patients had abnormal IgG waveform assays with both β₂GPI and prothrombin. The fourth patient (A006) had an abnormal IgG waveform assay with prothrombin only, but did have anti-β₂GPI antibodies detected by ELISA (FIG. 1). Three of these patients were also heterozygous for factor V Leiden (FIG. 1). Four patients had a single thromboembolic event and had: [1] abnormal IgG waveform assays in the absence of additional protein cofactors (A025, A445); [2] an abnormal assay with prothrombin only (A532); or [3] a normal IgG waveform assay (Al 25). Of note, one of these patients also had a second prothrombotic polymorphism (prothrombin G20210A polymorphism; patient A532). Only one patient in our study was a symptomatic (A005). This patient had an abnormal IgG waveform profile in the presence of prothrombin but not β₂GPI. None of the patients were homozygous for the non-phospholipid-binding β₂GPI polymorphisms at Cys³⁰⁶ or Trp³¹⁶ (FIG. 1), although patient A005 was heterozygous for Trp³¹⁶→Ser and patient A445 was heterozygous for Cys³⁰⁶→Gly.

The data above illustrate the ability to use a simple method based upon a routine coagulation laboratory test, the prothrombin time, or other coagulation reagent (APTT, TT, DRVVT, etc.) or a similar reagent that does not activate fibrin polymerization as set forth above, to detect the presence of APLA IgG antibodies. These examples demonstrate a method that can be used during warfarin oral anticoagulant therapy and which is not affected by heparin. Oral anticoagulant therapy is frequently monitored using the PT assay. Because different thromboplastins vary in sensitivity to plasma levels of factor II, VII and X, the international normalized ratio (INR) was introduced to allow comparison of PT times obtained with different reagents. PT clot time is often prolonged in patients with antiphospholipid syndrome (APS), which may add complexity in managing oral anticoagulant therapy in these patients, Furthermore, it has been shown that patients with APS who are receiving warfarin therapy often have greatly varied INRs that do not accurately reflect the true level of anticoagulation in those patients. Therefore, the use of INR to standardize PT is invalid for some patients with APS since high levels of antiphospholipid antibodies that might be present in the plasma may interfere with clot formation. At an anticoagulation therapy clinic, it is often difficult to determine which patient has APS and who does not, without going through a series of expensive testing. A PT slope 1 value from a routine PT test that is used to monitor anticoagulation therapy is therefore very useful in identifying patients with an increased likelihood of having APLA who may otherwise go unnoticed, or who may otherwise receive improper therapy. Oral anticoagulants can delay onset of coagulation but do not affect slope 1. Purified total IgG preparations from APLA patients not only produced negative slope 1, but also significantly prolonged the clot time and increased the INRs in IgG-depleted orally anticoagulated non-APLA plasma, suggesting a connection between increased INR value and the presence of APLA. Of course, reagents other than coagulation reagents, and analyzers other than coagulation analyzers can be used in the present invention.

More particularly, the above data shows the ability to identify antibody subsets that are biologically significant. Using an assay that employs purified patient IgG, purified protein co-factors and a specific thromboplastin that produced a negative PT slope 1 as set forth above, it has been possible to better define the components contributing to the abnormal PT waveform parameter and to recognize the potential application of the assay to identify patients at risk for recurrent thrombotic events.

The abnormal precoagulation phase detected in these patients was IgG antibody-mediated and is amplified by the presence of prothrombin and/or β₂GPI. APLA have been shown to bind to β₂GPI and prothrombin, and APLA-β₂GPI complexes as well as APLA-prothrombin complexes have been shown to bind to lipid membranes. It is possible that for some patients, other phospholipid-binding proteins may mediate this effect (e.g., protein S, high molecular weight kininogens), which may be the case for patient A125 in this study. These results also suggest that certain patients with antiprothrombin antibodies who are on warfarin therapy may be better detected by this assay if supplemental prothrombin is added to the reaction mixture.

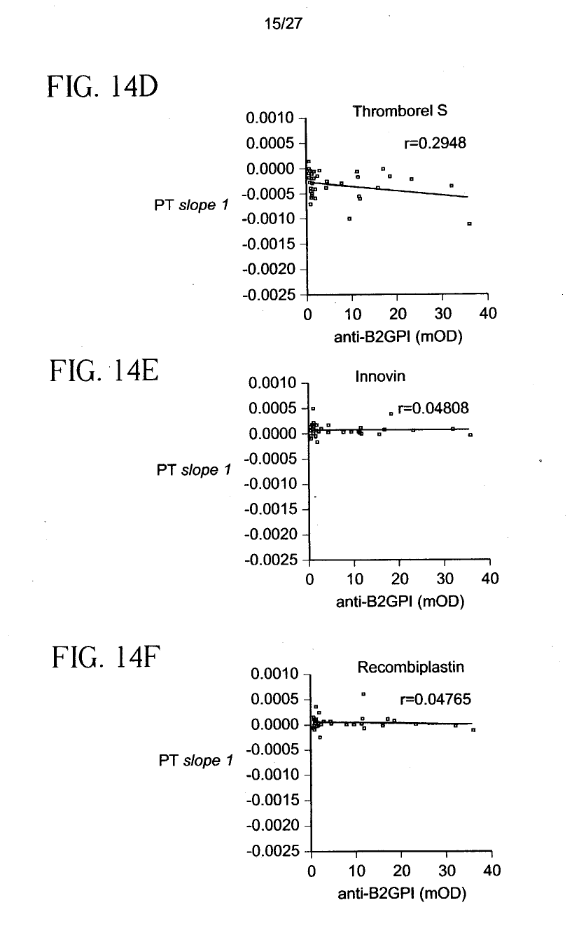

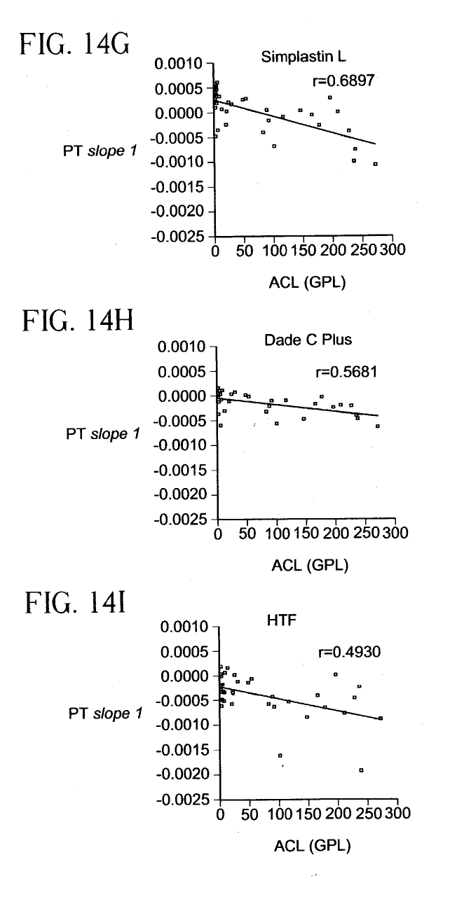

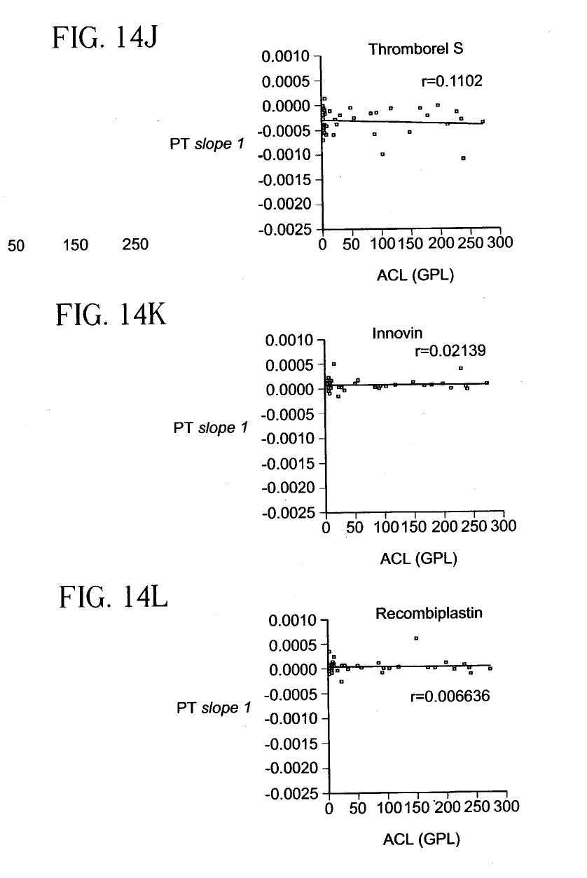

In separate experiments, not described above, it was shown that the abnormal IgG waveform results are not dependent on the presence of tissue factor. Dade Innovin® is composed of purified recombinant human tissue factor that is relipidated with mixtures of purified phosphatidylserine and phosphatidylcholine, which did not work as well as Simplastin L which is extracted from rabbit brain tissue and contains a complex mixture of phospholipids, including phosphatidylethanolamine, phosphatidylserine, phosphatidylcholine and other lipids. Other reagents that allowed for detection of APS individuals based on the slope_{—}1 determination, were Dade C plus and Simplastin R HTF. FIGS. 14A and 14B show the correlation of various reagents and different phospholipid binding proteins.

The data also shows that the IgG waveform assay can distinguish between pathological and non-pathological APLA. For example, those individuals with IgG that required β₂GPI to generate an abnormal IgG waveform profile had recurrent thrombotic problems (A003, A004 and A028). In contrast, one of three patients with IgG samples that demonstrated an abnormal waveform with prothrombin but not β₂GPI was asymptomatic (A005) and another had sustained a single venous thrombotic event (A532). The presence of additional prothrombotic risk factors (e.g., factor V Leiden) has also been shown to modify thrombotic risk in these patients, and three of the four patients with recurrent events were also heterozygous for factor V Leiden.

In the examples above, a PT reagent was added to each patient sample. However, it is also possible to add phospholipid vesicles to the individual's test sample instead of a PT reagent. The vesicles or liposomes can be, for example, purified phospholipids from natural sources, synthetic phospholipids, or platelets added to a test sample. If the phospholipids are from natural sources, the source can be mammalian tissue (e.g. brain tissue or placenta from a mammal—commonly rabbit). The phospholipids can be added to the test sample with or without a metal cation (commonly calcium or a calcium salt). If a standard PT reagent is not used vesicles or liposomes can be added in the form of platelets, cellular debris, phospholipids or platelet micro particles. In one embodiment of the invention one or more of PC, PS, PE or PI are added to the individual's test sample (with or without a metal cation) followed by optical monitoring of turbidity change in the test sample.

As mentioned above, because actual fibrin polymerization is not necessary for detecting the initial slope, activation of coagulation is not required. As is illustrated in FIG. 8, a slope 1 change is evident even when fibrin polymerization is inhibited by addition of a thrombin inhibitor. This type of waveform could also be achieved simply by mixing a reagent that does not activate coagulation (e.g., does not cause fibrin polymerization resulting in a clot) with the test sample. Without the onset of clot formation, slope 1 is determined as the slope of the waveform over a particular time period which time period can include a period that would have included clot formation had coagulation been allowed to occur. This possibly longer time period can allow for a greater number of data points over the longer period of time, potentially increasing the accuracy of the test in some situations.

In addition to phospholipids a metal cation can be added to the sample, though it is not needed to obtain the slope 1 or predict the APS condition. The metal cation is preferably a divalent metal cation, and can be added in the form of a salt. In a preferred embodiment, the salt is calcium chloride, though other salts (e.g., magnesium or manganese) could also be used. Buffers and stabilizers could also be added if desired. Any of the above components can be added separately or together as part of a non-coagulating reagent. Alternatively an inhibitor of thrombin could be added if a coagulation reagent is used, as mentioned above. If coagulation is not activated in the test, the overall drop in light transmittance (delta) could be used in a multi-parametric evaluation (at least slope 1 and delta).

If a coagulation reagent is used, then in addition to monitoring slope 1, as set forth herein, it is possible to utilize additional parameters from the clot waveform. The parameters of the profile can be one or more of time of initiation of clot formation, overall change in profile (e.g. total change in light transmittance), slope of profile after initiation of clot formation, acceleration at the time of clot initiation, slope after end of clot formation, etc. Preferred additional parameters are initiation of clot formation and slope after end of clot formation. The parameters having the greatest ability to distinguish APLA patients from normal patients are shown in Table 1 (PT waveforms).

A reagent or kit for performing the assay of the invention can include a coagulation activating reagent, particularly tissue factor as is found in a PT reagent. A preferred kit, however, comprises phospholipids in the form of phospholipid vesicles or liposomes as noted above, with or without a metal salt or metal ions. The kit also provides instructions for performing the assay and for determining whether the result of the assay indicates an increased likelihood of antiphospholipid antibodies in the sample. The instructions could also include a recommendation to seek confirmation (e.g. via immunoassay), or actual instructions for performing one or more confirmatory assays for confirming the antiphospholipid syndrome. If a coagulation reagent is used that comprises the phospholipids, directions should indicate determining slope 1 prior to initiation of clot formation. It is also possible to include a clot inhibitor in order to allow for determining a slope 1 over a greater period of time. Also, additional phospholipid binding protein may be added to enhance the assay's sensitivity, e.g. proteins to which APLA are specific (e.g. β₂ glycoprotein, cardiolipin, prothrombin), as well as instructions for addition of one or more of the proteins. Phospholipid binding proteins could be added to a PT reagent or to a reagent comprising phospholipid vesicles, followed by monitoring the clot profile. The phospholipid binding proteins could also be used in one or more confirmatory assays after a slope 1 is initially detected. In the confirmatory test, a particular phospholipid binding protein is added to the test sample along with the same reagent(s) from the initial test. If slope 1 becomes more severe, then the particular APLA antibody present is known. For example, if a test sample is tested and results in a slope 1, a second test can be run with the addition of, e.g. β₂ glycoprotein and/or prothrombin. If the second test results in a greater slope 1 than the first test, then the presence of antibody to the phospholipid biding protein (e.g. anti-β₂ glycoprotein) can be determined A kit can be provided having, not only phospholipids that can cause a slope_{—}1 for many patients with APS, but additionally one or more phospholipid binding proteins (prothrombin, β₂ glycoprotein, anticardiolipin) that can be added to the phospholipids in a confirmatory test. The kit instructions instruct the user to run an time dependent measurement profile by adding the kit phospholipids to a patient test sample (e.g. plasma). If a slope_{—}1 (e.g. beyond a particular value) results, then the kit user is instructed to perform a second assay where the phospholipids are added along with one or more of the phospholipid binding proteins to see whether the slope_{—}1 can be increased in the second assay. It is also possible to have a kit where the instructions indicate that, after a slope_{—}1 detection in a patient test sample, the amount of phospholipids should be increased in a subsequent assay in order to determine whether the slope_{—}1 value can be increased. And, of course, multiple additional assays (one or more assays where phospholipid binding proteins are added, and one or more assays where one or more phospholipids are increased in a subsequent assay).

Another confirmatory assay (and kit including the same) is a DRVVT test where dilute Russel's Viper Venom is added to a patient test sample to see whether clot time is prolonged and/or whether a slope_{—}1 results. It is also possible to run two DRVVT tests (one for screening and one for confirmation) where the amount of phospholipids is increased for the second test. If desired, an APTT can be run as the screening assay, and if a slope_{—}1 results that is beyond a particular threshold, then a DRVVT confirmatory assay is performed. In fact, a coagulation reagent (TT, PT, APTT, DRVVT etc.) or phospholipids can be used for the first screening assay, followed by the same or different reagent where the phospholipids are at a higher concentration. Or, a platelet neutralization assay can be performed as the confirmatory assay.

It is also possible to perform an APTT screening assay, and if a slope_{—}1 is present, perform a second modified APTT assay (same as standard APTT assay except without calcium) to rule out the possibility of the slope_{—}1 being caused by LCCRP. It is also possible to perform the modified APTT assay first, followed by an APTT assay (with calcium). The modified APTT assay can also be run on its own as the screening assay in the present invention, without a second assay.

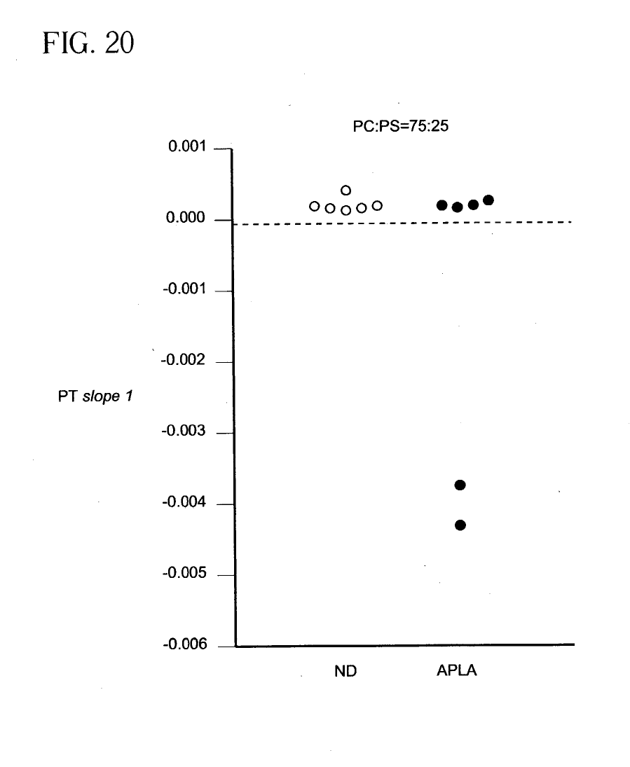

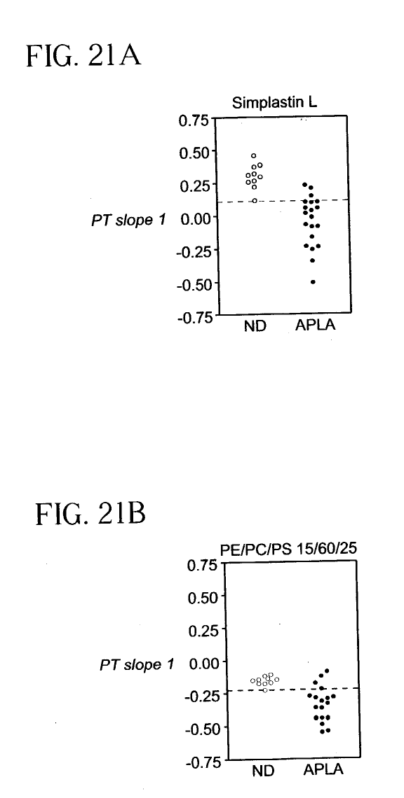

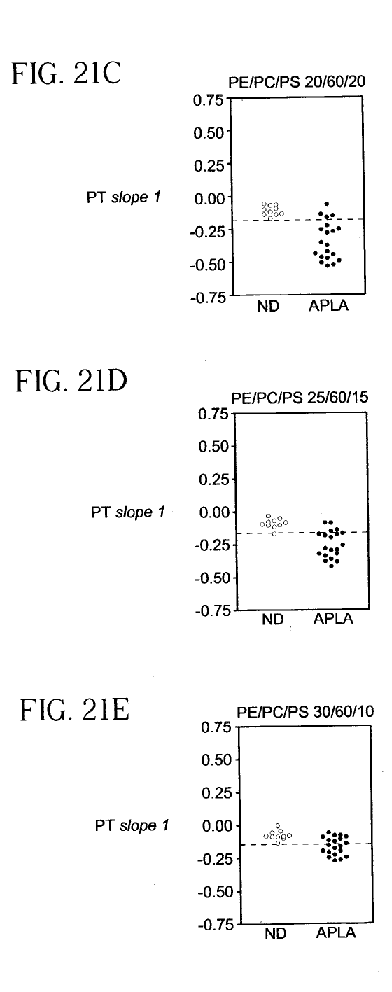

The phospholipids that can be used for the screening assay are preferably at least phosphatidylcholine (PC) and phosphatidylserine (PS), with optionally also phosphatidylethanolamine (PE) being part of the phospholipid mixture for increased sensitivity. The phospholipid mixture can comprise 10% or more of PS, preferably 15% or more. Amounts of 20% or more or 25% or more are also possible (10% to 30% being preferred). The PC amount in the phospholipid mixture is preferably at least 40% (preferably in the range of from 40 to 70%), whereas, in a mixture of PS, PC and PE, the remainder is PE—at least 5%, or at least 15% (e.g. in an amount of from 5 to 50% (preferably from 5 to 30%). Preferably the phospholipids are from natural sources though synthetically derived phospholipids can also be used. As can be seen in FIG. 20, a mixture of PC:PS=75:25 allows for detection of some APLA patients. However, as can be seen in FIG. 21, mixtures of PE/PC/PS achieve better discrimination between normals and APLA test samples.

If more than one assay is run on a patient test sample, the first phospholipid mixture can be selected to be at a lower concentration/amount than the phosopholipid mixture of the second test. The phospholipid mixture for the first test on the patient sample can be for making the first test highly sensitive, whereas the phospholipid mixture for the second test can be for making the second test more specific. Preferably the first (screening) test is with a sensitive phospholipid mixture or a prothrombin time reagent from natural sources.

In the examples above, a threshold is used (value for slope_{—}1) to predict the increased chance of a patient having APLA. The invention can also easily be practiced with multiple parameters and modeling, as disclosed in U.S. Pat. No. 6,101,449 to Givens et al. issued Aug. 8, 2000, and U.S. Pat. No. 6,321,164 to Braun et al. issued Nov. 20, 2001, mentioned hereinabove, as well as with self organizing feature maps as set forth in U.S. patent application Ser. No. 09/345,080 to Givens et al filed Jun. 30, 1999, each incorporated herein by reference. In one embodiment, one of the parameters of the model is slope prior to clot initiation (slope_{—}1) in the PT (or APTT or other coagulation reagent) profile. Other parts or parameters from the PT clot profile can also be used to predict an increased likelihood of APS. Referring again to Table 1, APLA patients not on an oral anticoagulant (warfarin) not only had a significantly different slope 1 from normal donors (as did APLA patients on warfarin), but also had significantly different clot time, tmin2, tmin1, tmax2, slope 3 and delta as compared to normals. These APLA patients also had significantly different clot time, slope 1, tmin2, tmin1 and tmax2 as compared to non-APLA patients on warfarin. APLA patients on warfarin not only had a significantly different slope 1 as compared to normal donors, but also had significantly different clot time, tmin2, min2, tmin1, tmax2, max2, slope 3 and delta as compared to normals. As such other parameters besides slope 1, or multiple parameters and modeling as set forth in U.S. Pat. No. 6,101,449 can be used to predict the existence of or an increased likelihood that a patient has APS.

Whether a single parameter threshold or a multi-parametric model is used, if there is an indication of the possibility of APLA in the patient sample, it may be desirable to run a confirmatory assay for APLA (e.g. an immunoassay) and/or an assay to distinguish from the possibility of LC-CRP (though a multi-parametric model may make this unnecessary). One such distinguishing assay that could be performed is an APTT assay with the addition of phosphorylcholine—a slope_{—}1 will not form in an APTT assay that originally had a slope_{—}1 due to LC-CRP. Or, if desired, a quantitative LC-CRP assay could be run to rule out the possibility of a slope_{—}1 caused by this mechanism (e.g. in an APTT assay). This might also be accomplished by adding a metal cation without phospholipids (e.g. calcium) or varying the type of coagulation reagent—if such is used to perform the assay (a reagent comprising phospholipids and a metal cation could be used in place of a coagulation reagent having phospholipids and a metal cation, as mentioned above). In any event, the existence of slope_{—}1 beyond a pre-determined threshold (or a model prediction that utilizes one or more parameters that could include slope_{—}1) is an indication of the possibility of APLA and should preferably be followed up by further testing to confirm whether or not the patient has APS.

Confirmatory assays for APLA can be one or more immunoassays for any of the (heterogenous) antiphospholipid antibodies. Preferably, the confirmatory assay is an immunoassay for anti-β₂ glycoprotein, anti-prothrombin or anticardiolipin antibody. Such immunoassays can be performed by any known assay method, such as metal sol immunoassays, ELISAs, latex immunoassays, etc. The confirmatory assay could also be an assay for identifying APLA according to the criteria: [1] prolongation of a phospholipid-dependent screening assay; [2] lack of correction of the prolonged assay with a 1:1 mix with pooled normal plasma; and [3] correction of the prolonged assay by the addition of excess phospholipid.

The assay of the present invention could also be a quantitative or semi-quantitative assay. As can be seen in FIGS. 14a and 14b, the degree of slope 1 can be correlated to an amount of antiphospholipid antibodies (in this case, anti-β₂ glycoprotein antibody and anticardiolipin antibody), degree of APS and/or probability of a thrombotic event. Correlation studies with antibody levels frequently elevated in patients with antiphospholipid antibodies revealed a relationship between a negative slope 1 value and the level of anticardiolipin IgG (r=0.7) as well as the level of antibodies to β₂ glycoprotein I (r=0.6) in this cohort of 66 APLA patients. In this way, APLA can be quantitated and/or the progression or regression of a patient can be monitored based on repeated tests for slope 1 (or based on repeated multi-parametric analyses as noted above).

In place of a PT reagent or phospholipids as set forth above, the reagent could be an APTT reagent. As can be seen in FIG. 3, slope 1 in an APTT clot profile can also indicate an increased possibility of a patient having antiphospholipid syndrome. And, as can be seen in Table 2, APLA patients not on an oral anticoagulant (warfarin) had significantly different APTT slope 1, as well as clot time, tmin2, tmin1, tmax2, max2, slope 3 and delta (as compared to normal) from the APTT clot profile. These APLA patients not on oral anticoagulant also had significantly different slope 1 and slope 3 as compared to non-APLA patients on oral anticoagulant. APLA patients on oral anticoagulant had significantly different slope 1 as well as slope 3 as compared to non-APLA patients on oral anticoagulant, and significantly different APTT clot time, tmin2, min2, tmin1, tmax2, max2 and delta as compared to the normals. These parameters, alone or together in a multi-parametric model (e.g. a neural network model as mentioned earlier), could also be used to predict an increased likelihood of APS.

The invention is also directed to determining which patients are acute risk patients, such as those that are at an increased risk of a thrombotic event. Thrombosis is the clinical event that is most commonly associated with the presence of antiphosholipid antibodies. Thrombotic events are reported in up to 30% of patients with antiphospholipid antibodies, with an overall incidence of 2.5 patients per 100 patient-years. Venous thromboembolism (VTE) accounts for about two thirds of the thrombotic events. Stroke is the most prevalent arterial occlusive event, often occurring at a young age. Also recurrence rates of thrombosis are particularly high and the presence of APA's is further linked to poor functional prognosis, including organ damage and increased risk of cardiovascular disease. Due to the high incidence of thrombosis in individuals with antiphospholipid antibodies, the invention herein can also be a test where phospholipids are added to a test sample, a time dependent measurement is taken, and a slope_{—}1 is determined—and if the slope_{—}1 value is beyond a particular value, then it is determined that the individual is at an increased risk of experiencing a thrombotic event.

In a variation of the above, an individual is determined to be at an increased risk of a thrombotic event, where a first test is performed where phospholipids are added to a patient test sample and a slope_{—}1 beyond a particular threshold is detected. Then a second test is performed where phospholipids and either beta 2 glycoprotein I or prothrombin is added to a patient test sample. If an increased slope_{—}1 is observed when adding additional beta 2 glycoprotein I (or if an increased slope_{—}1 is not observed when adding prothrombin) then there is an increased likelihood that the patient's APS is due to the presence of elevated levels of beta 2 glycoprotein 1, which has been shown to be associated with an increased risk for thrombotic complications compared to APS associated with antiprothrombin antibodies. This method can also be used to determine which APS patients are at a higher risk of experiencing a clinical manifestation of APS (e.g. miscarriage, thrombotic event, SLE, autoimmune disorder, etc.) based on the existence of (and/or degree of) slope_{—}1 in the clot profile.

The invention can also be applied for determining which individuals are at an increased risk of experiencing a miscarriage, and/or for determining that the cause of an already-experienced miscarriage was due to APS. In such a method, a test sample from an individual is provided; the test sample is combined with phospholipids; a light beam is directed at the test sample and light scattering or transmittance is monitored over time so as to provide a time-dependent measurement profile; a value or a slope is detected at or over a particular time in the time-dependent measurement profile that is beyond a corresponding predetermined value or slope threshold; and if the value or slope in the time-dependent measurement profile is beyond the predetermined threshold, then it is determined that the individual has an increased risk of experiencing a miscarriage (or that there is a likelihood that an already-experienced miscarriage was due to APS).

The invention can also be used to monitor the condition of individuals who have been determined to be at an increased risk of APS (or who have been confirmed as being APS patients), where the test is performed multiple times every few weeks or months or over other intervals. If APS patients are treated with a drug such as LJP 1082 (from La Jolla Pharmaceutical Co.) that targets anti-beta 2 glycoprotein I—or another drug that targets this or other antibodies to phospholipid binding proteins, then such therapy can be monitored over time by determining the existence (and degree) of the slope_{—}1 from assays such as described hereinabove.

The invention is also directed to determining an increased likelihood of systemic lupus erythematosus (SLE). SLE is one of the most frequent conditions, reported in 35% of patients with antiphospholipid antibodies. SLE accounts for more than 100,000 hospital admissions in the United States each year, and SLE is a leading cause of kidney disease and stroke in women of child bearing age.

The methods of the invention need not be performed on a coagulation analyzer, but can also be performed on a clinical chemistry analyzer or other machine that allows for determining a change in sample turbidity (or viscosity) over time, preferably one that allows for monitoring light transmittance through a sample. When the slope_{—}1 beyond a particular value is determined in accordance with the above, the sample can be flagged as being a likely APS sample. Such flagging can be by an alert on a printer in communication with the analyzer/apparatus, or on a monitor/screen, audio alert, etc.

Although the present invention has been shown and described with reference to the preferred embodiments, those skilled in the art will appreciate that various modifications and changes in form and details may be made without departing from the spirit and scope of the invention. For example, in the preceding description specific details are set forth to provide a more thorough understanding of the invention, but it will be apparent to those skilled in the art that the invention may be practiced without using these specific details.

We claim:

1. A method for predicting that an individual has an increased likelihood of having antiphospholipid syndrome, comprising:
a) providing a test sample from an individual;
b) combining the test sample with phospholipids;
c) directing a light beam at the test sample and monitoring light scattering or transmittance over time so as to provide a time-dependent measurement profile;
d) determining if a value or a slope at or over a particular time in the time-dependent measurement profile is beyond a corresponding predetermined value or slope threshold; and
if the value or slope in the time-dependent measurement profile is beyond the predetermined threshold, then determining that the individual has an increased likelihood of antiphospholipid syndrome.

2. The method according to claim 1, wherein the time-dependent measurement is an optical measure of changes in absorbance and/or transmittance through the sample over time.

3. The method according to claim 1, wherein the sample is a whole blood or plasma sample from the individual.

4. The method according to claim 1, wherein the phospholipids are added as part of a coagulation reagent that comprises thromboplastin.

5. The method according to claim 4, wherein the coagulation reagent is a prothrombin time reagent.

6. The method according to claim 1, wherein the phospholipids are a heterogenous mixture of phospholipids of varying structures, including non-bilayer arrangements.

7. The method according to claim 1 wherein the phospholipids are from natural sources.

8. The method according to claim 4, wherein the coagulation reagent comprises tissue factor, a halide salt and a mixture of phospholipids.

9. The method according to claim 1, further comprising adding a divalent metal cation or a salt of a divalent metal cation along with the phospholipids.

10. The method according to claim 1, further comprising performing at least one confirmatory assay to determine the existence of antiphospholipid antibodies.

11. The method according to claim 10, wherein the at least one confirmatory assay is a latex immunoassay or an ELISA.

12. The method according to claim 10, wherein the at least one immunoassay comprises an assay for at least one of anti-β₂ glycoprotein, anti-prothrombin and anticardiolipin antibodies.

13. The method according to claim 1, wherein the individual is a person taking an oral anticoagulant.

14. The method according to claim 10, further comprising initiating oral anticoagulant therapy if antiphospholipid antibodies are found in the confirmatory assay.

15. The method according to claim 1, wherein the time-dependent measurement profile is an optical transmittance profile.

16. The method according to claim 15, wherein the optical transmittance profile is generated on a photo-optical coagulation analyzer.

17. The method according to claim 1, wherein the individual is one who has experienced spontaneous miscarriage or a thromboembolic event.

18. The method according to claim 1, further comprising performing an APTT assay on a sample from the individual to determine whether the APTT exhibits a prolonged clot time.

19. The method according to claim 10, wherein the at least one confirmatory assay is confirmatory assay for identifying APLA according to the criteria: a) prolongation of a phospholipid-dependent screening assay; b) lack of correction of the prolonged assay with a 1:1 mix with pooled normal plasma; and c) correction of the prolonged assay by the addition of excess phospholipid.

20. The method according to claim 1, wherein the phospholipids are not added as part of a coagulation reagent.

21. The method according to claim 1, wherein a confirmatory assay is run which comprises deriving a time-dependent measurement profile with an APTT reagent and determining if there is a negative slope 1 in the time-dependent measurement.

22. The method according to claim 1, wherein a confirmatory assay is run that is a platelet neutralization test.

23. The method of claim 21, wherein if there is no slope 1 in the APTT time-dependent measurement beyond a predetermined value or threshold, then performing an additional confirmatory assay which is an immunoassay.

24. The method of claim 23, wherein the immunoassay is an ELISA for anti-β₂ glycoprotein, anti-prothrombin and anticardiolipin antibodies.

25. The method of claim 1, wherein the vesicles or liposomes are part of a DRVVTDRVVT reagent that comprises Russel's Viper Venom.

26. The method of claim 1, wherein if the value or slope is beyond a predetermined threshold, then determining if the test sample comprises C-reactive protein or LC-CRP.

27. The method of claim 26, wherein determining if the test sample comprises C-reactive protein comprises performing an APTT assay in the presence and absence of phosphorylcholine.

28. The method of claim 27, wherein if there is a negative APTT slope and this is inhibited by phosphorylcholine, performing a confirmatory test for APLA.

29. The method of claim 1, wherein the phospholipids are phosphatidylethanolamine, phosphatidylcholine, phosphatidylserine and/or phosphatidylinositol.

30. The method of claim 29, further comprising adding a metal cation in the form of a metal salt prior to determining the slope or value.

31. The method of claim 29, wherein a plurality of phosphatidylethanolamine, phosphatidylcholine, phosphatidylserine and phosphatidylinositol are added to the test sample.

32. The method of claim 1, wherein the test sample is purified IgG from the individual.

33. The method of claim 32, further comprising adding a phospholipid binding protein to the test sample prior to determining the value or slope.

34. The method of claim 33, wherein the phospholipid binding protein is 2 glycoprotein I, cardiolipin or prothrombin.

35. The method of claim 33, further comprising adding a coagulation reagent to the test sample prior to determining the value or slope.

36. The method of claim 1, wherein the individual is not a disseminated intravascular coagulation patient.

37. The method of claim 1, wherein the phospholipids are part of a reagent derived from mammal tissue.

38. The method of claim 37, wherein the tissue is brain or placenta.

39. The method of claim 1, wherein the phospholipids are added in the absence of a source of metal cation.

40. The method of claim 1, wherein the phospholipids are part of a prothrombin time reagent.

41. The method of claim 1, further comprising performing the method of claim 1 again with phospholipids more sensitive to APLA.

42. The method of claim wherein the at least one immunoassay comprises an assay for anti-β₂ glycoprotein.

43. The method of claim 12, wherein the at least one immunoassay comprises an assay for anti-prothrombin.

44. The method of claim 12, wherein the at least one immunoassay comprises an assay for anti-β₂ glycoprotein and anti-prothrombin.

45. The method of claim 12, wherein the at least one immunoassay comprises an assay for anticardiolipin antibodies.

46. The method of claim 1, wherein the test sample is from an individual on oral anticoagulant, and wherein prothrombin is added to the test sample along with phospholipids.

47. The method according to claim 1, further comprising performing a confirmatory assay to determine the existence of phospholipid binding proteins.

48. The method of claim 47, wherein the confirmatory assay is an assay for β₂ glycoprotein, prothrombin or anticardiolipin.

49. The method of claim 1, wherein a test sample is combined with phospholipids and a time dependent measurement profile is obtained in the absence of adding a coagulation reagent to the test sample.

50. The method of claim 1, wherein a confirmatory assay is performed after determining an increased likelihood of the presence of antiphospholipid antibodies, the confirmatory assay comprising a) prolongation of a phospholipid-dependent screening assay; b) lack of correction of the prolonged assay with a 1:1 mix with pooled normal plasma; and c) correction of the prolonged assay by the addition of excess phospholipid.

51. The method of claim 1, wherein if a slope_{—}1 beyond a predetermined threshold is detected, a confirmatory assay is performed after determining an increased likelihood of the presence of antiphospholipid antibodies, the confirmatory assay comprising adding phospholipids to a test sample from the individual along with at least one prothrombin binding protein, performing a time dependent measurement profile on the sample, and determining whether there is an increase or not in the slope_{—}1 compared to the initial slope_{—}1 detected.

52. The method of claim 1, wherein the phospholipids added are not part of a PT or APTT reagent.

53. The method of claim 1, further comprising, if a slope_{—}1 is detected as being beyond a predetermined threshold, performing the method of claim 1 again with the addition of one phospholipid binding protein and performing the method of claim 1 yet again with the addition of another phospholipid binding protein, and determining whether there is an increase in the slope_{—}1 for each additional test.

54. The method of claim 1, further comprising, if a slope_{—}1 is detected as being beyond a predetermined threshold, performing a DRVVT test as a confirmatory test.

55. The method of claim 1, wherein the phospholipids comprise PC and PS.

56. The method of claim 55, wherein the phospholipids further comprise PE.

57. The method of claim 56, wherein PS is 10% or more of the total phospholipids.

58. The method of claim 54, wherein the PS is 15% or more of the total phospholipids.

59. The method of claim 58, wherein the PS is 25% or more of the total phospholipids.

60. The method of claim 56, wherein the PC is at least 40% of the total phospholipids.

61. The method of claim 60, wherein the PC is at least 60% of the total phospholipids.

62. The method of claim 61, wherein the PC is from 40% to 70% of the total phospholipids.

63. The method of claim 56, wherein the PE is at least 5% of the total phospholipids.

64. The method of claim 63, wherein the PE is at least 15% of the total phospholipids.

65. The method of claim 63, wherein the PE is from 5 to 50% of the total phospholipids.

66. The method of claim 65, wherein the PE is from 5 to 30% of the total phospholipids.

67. The method of claim 56, wherein the PS is from 10% to 30%, PC is from 40% to 70% and PE is from 5% to 50% of the total phospholipids.

68. The method according to claim 4, wherein the coagulation reagent is a thrombin time reagent.

69. The method according to claim 4, wherein the coagulation reagent is a dilute Russel's Viper Venom reagent.

70. The method according to claim 4, wherein the coagulation reagent is a activated partial thromboplastin time reagent.

71. The method according to claim 4, wherein the coagulation reagent is a reagent comprising snake venom and phospholipids.

72. The method of claim 1, wherein the phospholipids are phospholipids sufficient to cause a slope_{—}1 beyond a predetermined threshold in a majority of patients with antiphospholipid syndrome.

73. The method of claim 1, wherein if it is determined that a value or a slope at or over a particular time in the time-dependent measurement profile is beyond a corresponding predetermined value or slope threshold, then the test sample is flagged as being a sample from an individual with an increased likelihood of having antiphospholipid syndrome.

74. The method of claim 73, wherein the flagging is performed by printing an alert on a printer in communication with an analyzer on which the method is performed.

75. The method of claim 73, wherein the flagging is performed by displaying an alert on a monitor in communication with an analyzer on which the method is performed.

76. A method for predicting that an individual is at an increased likelihood for having antiphospholipid syndrome, comprising:
a) providing a test sample from an individual;
b) combining the test sample with a coagulation reagent comprising phospholipids;
c) monitoring the formation of fibrin polymerization over time so as to provide a time-dependent measurement profile;
d) defining a set of one or more predictor variables which sufficiently define the data of the time-dependent measurement profile;
e) deriving a model that represents the relationship between the antiphospholipid syndrome and the one or more predictor variables; and
f) utilizing the model of step e) to predict the increased likelihood of antiphospholipid syndrome in the individual.

77. The method according to claim 76, wherein the coagulation reagent is a PT reagent.

78. The method according to claim 77, further comprising repeating steps a) to e) with an APTT reagent.

79. The method according to claim 78, wherein when the PT reagent is used the one or more predictor variables includes slope prior to clot initiation, slope post coagulation and time of initiation of clot formation.

80. The method according to claim 79, wherein when the APTT reagent is used, the one or more predictor variable include one or more of time corresponding to coagulation onset, time corresponding to midpoint of coagulation, time corresponding to end of coagulation, and value or slope prior to clot initiation.

81. The method according to claim 76, wherein the predictor variables include clot time or INR, and slope prior to initiation of clot formation.

82. The method of claim 76, wherein the coagulation reagent comprising the phospholipids is a prothrombin time reagent.

83. The method of claim 82, wherein the one or more parameters are slope 1, tmin2, tmin1, tmax2, slope 3 and delta.

84. The method of claim 83, wherein the individual is an individual not on oral anticoagulant therapy.

85. The method of claim 82, wherein the one or more parameters are slope 1, tmin2, tmin1 and tmax2.

86. The method of claim 82, wherein the one or more parameters are slope 1, tmin2, min2, tmin1, tmax2, max2, slope 3 and delta.

87. The method of claim 86, wherein the individual is an individual on oral anticoagulant therapy.

88. The method of claim 76, wherein the time dependent measurement profile is at least one optical profile.

89. The method of claim 88, wherein the optical profile is provided by an automated analyzer for thrombosis and hemostasis testing.

90. The method of claim 76, wherein after step f, one or more assays for confirming the predicted antiphospholipid syndrome are performed.

91. The method of claim 90, wherein the one or more confirmatory assays are one or more immunoassays for antiphospholipid antibodies.

92. The method of claim 76, wherein the model is a neural network.

93. The method of claim 76, wherein the phospholipids are phosphatidylethanolamine, phosphatidylcholine, phosphatidylserine and/or phosphatidylinositol.

94. The method of claim 93, further comprising adding a metal cation in the form of a metal salt prior to determining the slope or value.

95. The method of claim 93, wherein a plurality of phosphatidylethanolamine, phosphatidylcholine, phosphatidylserine and phosphatidylinositol are added to the test sample.

96. The method of claim 76, wherein the test sample is purified IgG from the individual.

97. The method of claim 96, further comprising adding a phospholipid binding protein to the test sample prior to determining the value or slope.

98. The method of claim 97, wherein the phospholipid binding protein is 2 glycoprotein I, cardiolipin or prothrombin.

99. The method of claim 97, wherein the phospholipids are added to the test sample as part of a coagulation reagent prior to determining the value or slope.

100. The method of claim 76, wherein the individual is not a disseminated intravascular coagulation patient.

101. The method of claim 76, wherein the phospholipids are part of a reagent derived from mammal tissue.

102. The method of claim 101, wherein the tissue is brain or placenta.

103. The method of claim 76, wherein the phospholipids are added in the absence of a source of metal cation.

104. The method of claim 76, wherein the phospholipids are part of a prothrombin time reagent.

105. The method of claim 91, wherein the one or more immunoassays are immunoassays for anti-β₂ glycoprotein, anti-prothrombin and/or anticardiolipin antibodies.

106. The method of claim 1, further comprising performing a second assay on those test samples that indicate that an individual is at an increased likelihood of having antiphospholipid syndrome, wherein the results of the second assay are capable of increasing the likelihood of the individual having antiphospholipid syndrome.

107. A method for predicting antiphospholipid syndrome in an individual from at least one time-dependent measurement profile, comprising:
a) combining a test sample from an individual with phospholipids and directing a light beam at a test sample and monitoring light scattering or transmittance over time so as to provide a time-dependent measurement profile;
b) defining a set of one or more predictor variables which sufficiently define the data of the time-dependent measurement profile;
c) deriving a model that represents the relationship between the antiphospholipid syndrome and the one or more predictor variables; and
d) utilizing the model of step c) to predict the existence of antiphospholipid syndrome in the individual.

108. The method according to claim 107, wherein the one or more predictor variables includes a decrease in value or a slope of the profile prior to clot initiation.

109. The method according to claim 108, wherein the one or more predictor variables is a plurality of variables that further includes one or more of: a minimum of the first derivative of the profile, a time index of the minimum of the first derivative, a minimum of the second derivative of the profile, a time index of the minimum of the second derivative, a maximum of the second derivative of the profile, a time index of the maximum of the second derivative, an overall change in the coagulation parameter during the time-dependent measurement on the unknown sample, a clotting time, and a slope of the profile after clot formation.

110. The method according to claim 109, wherein said at least one time-dependent measurement profile is at least one optical profile.

111. The method according to claim 110, wherein said at least one optical profile is provided by an automated analyzer for thrombosis and hemostasis testing.

112. The method according to claim 111, wherein a plurality of optical measurements at one or more wavelengths are taken over time so as to derive said at least one optical profile, said optical measurements corresponding to changes in light scattering and/or light absorption in the unknown sample.

113. The method according to claim 112, wherein a plurality of optical measurements are taken over time so as to derive said at least one optical profile, and wherein said plurality of optical measurements are each normalized to a first optical measurement.

114. The method according to claim 110, wherein in step a) said at least one optical profile is provided automatically by said analyzer, whereby said sample is automatically removed by an automated probe from a sample container to a test well, one or more reagents are automatically added to said test well so as to initiate said property changes within said sample, and the development of said property over time is automatically optically monitored so as to derive said optical data profile.

115. The method according to claim 114, wherein after step d), a predicted antiphospholipid syndrome is automatically stored in a memory of said automated analyzer and/or displayed on said automated analyzer.

116. The method according to claim 114, wherein after step d), one or more assays for confirming the predicted antiphospholipid syndrome are performed.

117. The method according to claim 116, wherein the one or more confirmatory assays comprise one or more immunoassays for antiphospholipid antibodies.

118. The method according to claim 107, wherein said model of step c) is a neural network.

119. The method according to claim 107, wherein said relationship in step c) is determined via at least one automated algorithm.

120. The method according to claim 119, wherein said model is a multilayer perceptron, and wherein said at least one algorithm is a back propagation learning algorithm.

121. The method according to claim 107, wherein in step a), a plurality of time-dependent measurement profiles are derived for use in step b).

122. The method according to claim 121, wherein said plurality of time dependent measurement profiles includes at least two profiles from assays initiated with PT reagents, APTT reagents, fibrinogen reagents and TT reagents.

123. The method according to claim 107, wherein the sample is a sample from a medical patient, and wherein in step d), both said model and additional patient medical data are utilized for predicting antiphospholipid syndrome in the individual.

124. The method according to claim 116, wherein the one or more confirmatory assays is a confirmatory assay for identifying APLA according to the criteria: a) prolongation of a phospholipid-dependent screening assay; b) lack of correction of the prolonged assay with a 1:1 mix with pooled normal plasma; and c) correction of the prolonged assay by the addition of excess phospholipid.

125. A method for predicting an increased risk of thrombosis in a test subject, comprising:
a) providing a test sample from an individual;
b) combining the test sample with phospholipids;
c) directing a light beam through the test sample and monitoring the transmittance of light through the sample over time so as to provide a time-dependent measurement profile;
d) determining if a value or slope in the time-dependent measurement profile at a particular time is beyond a corresponding predetermined value or slope threshold; and
if the value or slope in the time-dependent measurement profile is beyond the predetermined threshold, then determining an increased risk of thrombosis in the test subject.

126. The method according to claim 125, wherein the light beam is from a monochromatic light source in an automated coagulometer.

127. The method according to claim 125, wherein the sample is a whole blood or plasma sample from the individual.

128. The method according to claim 125, wherein the phospholipids are part of a coagulation reagent comprising thromboplastin.

129. The method according to claim 128, wherein the coagulation reagent is a PT reagent.

130. The method according to claim 129, wherein the phospholipids are vesicles or liposomes.

131. The method according to claim 125, wherein the phospholipids are not added to the test sample as part of a coagulation reagent.

132. The method according to claim 125, wherein the phospholipids are part of a coagulation reagent that comprises thromboplastin, and a halide salt.

133. The method according to claim 129, further comprising adding a metal cation or a salt of a metal cation.

134. The method according to claim 125, further comprising performing at least one confirmatory assay to determine the existence of antiphospholipid antibodies.

135. The method according to claim 134, wherein the at least one assay is a latex immunoassay or an ELISA.

136. The method according to claim 134, wherein the at least one confirmatory assay is an assay for anti-β₂ glycoprotein, anti-prothrombin and anticardiolipin antibodies.

137. The method according to claim 125, wherein the individual is a patient taking an oral anticoagulant.

138. The method according to claim 134, further comprising treating the individual with an oral anticoagulant if antiphospholipid antibodies are determined.

139. The method according to claim 125, wherein the time-dependent measurement profile is an optical transmittance profile.

140. The method according to claim 139, wherein the optical transmittance profile is generated on a photo-optical coagulation analyzer.

141. The method according to claim 125, wherein the individual is one who has experienced spontaneous miscarriage or a thromboembolic event.

142. The method according to claim 125, further comprising performing an APTT assay on a sample from the individual to determine whether the APTT exhibits a prolonged clot time.

143. A method for monitoring the therapy of an individual having antiphospholipid syndrome, comprising:
a) providing a test sample from an individual with APS;
b) combining the test sample with phospholipids;
c) directing light at the test sample and monitoring light reflectance from or transmittance through the test sample over time so as to provide a time-dependent measurement profile;
d) determining a value or slope in the time-dependent measurement profile;
e) administering therapy to the individual;
f) repeating steps a) to d); and
g) comparing the values or slopes to each other in order to determine the efficacy of said therapy.

144. The method according to claim 143, wherein the therapy is the administration of an oral anticoagualant.

145. The method according to claim 143, wherein if the value decreases overtime or the slope increases over time, then it is determined that the individual's condition is worsening, and if the value increases over time or the slope decreases over time, then it is determined that the individual's condition is improving.

146. The method according to claim 143, wherein the vesicles or liposomes are added as part of a coagulation reagent.

147. The method according to claim 146, wherein the coagulation reagent is a DRVVT or PT reagent.

148. The method according to claim 146, wherein the INR of the sample is determined.

149. The method according to claim 148, wherein the value or slope and the INR are used to manage the therapy of the individual.

150. The method of claim 143, wherein the therapy is directed at reducing APLA antibodies.

151. A method for monitoring the therapy of an individual having antiphospholipid syndrome, comprising:
a) providing a test sample from an individual with APS;
b) combining the test sample with a coagulation reagent, and added phospholipids
c) monitoring the formation of fibrin polymerization over time so as to provide a time-dependent measurement profile;
d) determining a value or slope in the time-dependent measurement profile prior to initiation of clot formation;
e) administering therapy to the individual based on the value or slope determined.

152. The method according to claim 151, wherein the further the value or slope is beyond threshold, the greater the therapy provided to the individual.

153. The method according to claim 152, wherein the therapy is the administration of oral anticoagulant, and wherein the dosage of the oral anticoagulant is increased or decreased depending upon the value or slope determined.

154. A method for categorizing an individual as an acute risk individual within a population of APS individuals, comprising:
a) providing a test sample from an individual;
b) combining the test sample with phospholipids;
c) directing a light beam at the test sample and monitoring light scattering or transmittance over time so as to provide a time-dependent measurement profile;
d) determining if a value or slope at a particular time in the time-dependent measurement profile is beyond a corresponding predetermined value or slope threshold; and
if the value or slope in the time-dependent measurement profile is beyond the predetermined threshold, then determining that the APS individual is an acute risk individual.

155. The method according to claim 154, wherein the acute risk is an acute risk of a thrombotic event.

156. The method according to claim 154, wherein the acute risk is an acute risk of a miscarriage, SLE or autoimmune disorder.

157. The method according to claim 156, wherein the acute risk is an acute risk of SLE.

158. The method of claim 154, wherein the acute risk is an acute risk of thrombocytopenia.

159. A method for indirectly measuring a level of antiphospholipid antibodies in a test sample from a test subject with antiphospholipid syndrome, comprising:
a) providing a test sample from an individual;
b) combining the test sample with phospholipids;
c) directing a light beam at the test sample and monitoring light scattering or transmittance over time so as to provide a time-dependent measurement profile;
d) determining the value or slope at a particular time in the time-dependent measurement profile; and
correlating the value or slope to a level of antiphospholipid antibodies in the test sample.

160. The method of claim 159, wherein the antiphospholipid antibodies are anti-β₂ glycoprotein, anti-prothrombin and/or anticardiolipin antibody.

161. The method of claim 160, wherein the level of anti-β₂ glycoprotein or anticardiolipin is determined.

162. A method for predicting that an individual is at an increased likelihood for having antiphospholipid syndrome, comprising:
a) providing a test sample from an individual;
b) combining the test sample with an APTT reagent;
c) monitoring the formation of fibrin polymerization over time so as to provide a time-dependent measurement profile;
d) defining a set of one or more predictor variables which sufficiently define the data of the time-dependent measurement profile;
e) deriving a model that represents the relationship between the antiphospholipid syndrome and the one or more predictor variables; and
f) utilizing the model of step e) to predict the increased likelihood of antiphospholipid syndrome in the individual.

163. The method of claim 162, wherein the time dependent measurement profile is at least one optical profile.

164. The method of claim 163, wherein the optical profile is provided by an automated analyzer for thrombosis and hemostasis testing.

165. The method of claim 162, wherein after step f, one or more assays for confirming the predicted antiphospholipid syndrome are performed.

166. The method of claim 165, wherein the one or more confirmatory assays are one or more immunoassays for antiphospholipid antibodies.

167. The method of claim 162, wherein the model is a neural network.

168. The method of claim 162, wherein the one or more parameters are selected from slope 1, clot time, tmin2, min2, tmin1, min1, tmax2, max2, slope 3 and delta.

169. The method of claim 168, wherein a plurality of parameters are used and are selected from slope 1, clot time, tmin2, tmin1, tmax2, max2, slope 3 and delta.

170. The method of claim 169, wherein the individual is not on oral anticoagulant therapy.

171. The method of claim 168, wherein a plurality of parameters are used, at least two of which are slope 1 and slope 3.

172. The method of claim 162, further comprising performing one or more confirmatory assays.

173. The method of claim 172, wherein the one or more confirmatory assays included assaying for C reactive protein or LC-CRP.

174. The method of claim 173, wherein the one or more confirmatory assays comprises an immunoassay for at least one antiphospholipid antibody.

175. The method of claim 174, wherein the antiphospholipid antibody is anti-β₂ glycoprotein, anti-prothrombin and/or anticardiolipin antibody.

176. The method of claim 162, wherein the individual is on oral anticoagulant therapy.

177. The method of claim 162, further comprising performing a PT assay on a test sample from the individual and determining whether there is a slope 1 beyond a predetermined threshold.

178. The method of claim 168 further comprising running a second assay with an APTT reagent and phophorylcholine to determine whether slope 1 is inhibited.

179. The method of claim 162, wherein a single parameter is used and the model is a threshold.

180. A method for determining an increased risk of antiphospholipid syndrome, comprising:
a) adding an APTT reagent to an individual's test sample,
b) performing a time dependent measurement profile on the test sample,
c) determining whether the profile exhibits a slope or value beyond a predetermined threshhold prior to initiation of clot formation, and if so,
d) repeating steps (a) to (c) except with an APTT reagent not comprising calcium so as to confirm the determination of antiphospholipid syndrome (or an increased likelihood of antiphospholipid syndrome) is the profile again exhibits a slope or value beyond a predetermined threshold.

181. A method for monitoring an individual, comprising:
a) providing a test sample from an individual;
b) combining the test sample with phospholipids;
c) directing a light beam at the test sample and monitoring light scattering or transmittance over time so as to provide a time-dependent measurement profile;
d) determining if a value or a slope at or over a particular time in the time-dependent measurement profile is beyond a corresponding predetermined value or slope threshold; and
if the value or slope in the time-dependent measurement profile is beyond the predetermined threshold, then determining that the individual has antiphospholipid syndrome or an increased risk of antiphospholipid syndrome.

182. A method for detecting antiphospholipid antibodies in a test sample, comprising:
a) providing a test sample from an individual;
b) combining the test sample with phospholipids;
c) directing a light beam at the test sample and monitoring light scattering or transmittance over time so as to provide a time-dependent measurement profile;
d) determining if a value or a slope at or over a particular time in the time-dependent measurement profile is beyond a corresponding predetermined value or slope threshold; and
if the value or slope in the time-dependent measurement profile is beyond the predetermined threshold, then determining that the individual has antiphospholipid antibodies.

183. A method for determining an increased likelihood that an individual will experience a clinical event due to an underlying APS, comprising:
a) providing a test sample from an individual;
b) combining the test sample with phospholipids;
c) directing a light beam at the test sample and monitoring light scattering or transmittance over time so as to provide a time-dependent measurement profile;
d) determining if a value or a slope at or over a particular time in the time-dependent measurement profile is beyond a corresponding predetermined value or slope threshold; and
if the value or slope in the time-dependent measurement profile is beyond the predetermined threshold, then determining that the individual has an increased likelihood of experiencing a clinical event due to underlying APS.

184. The method of claim 183, wherein the clinical event is SLE, miscarriage, thrombosis or an autoimmune disorder.
